(19) United States
(12) Patent Application Publication
(10) Pub. No.: US 2022/0375609 A1
(43) Pub. Date: Nov. 24, 2022

(54) CLINICAL VARIANT CLASSIFIER MODELS, MACHINE LEARNING SYSTEMS AND METHODS OF USE

(71) Applicant: NemaMetrix, Inc, Eugene, OR (US)

(72) Inventors: Lan Guo, Eugene, OR (US); Stephen Turner, Eugene, OR (US); Trisha Brock, Eugene, OR (US); Chris HOPKINS, Corvallis, OR (US)

(73) Assignee: NemaMetrix, Inc., Eugene, OR (US)

(21) Appl. No.: 17/769,727

(22) PCT Filed: Oct. 15, 2020
(86) PCT No.: PCT/US20/55828

§ 371 Date: Apr. 15, 2022

Related U.S. Application Data
(60) Provisional application No. 62/916,141, filed on Oct. 16, 2019.
(60) Provisional application No. 62/952,219, filed on Dec. 21, 2019.

(51) Int. Cl. G16H 50/20; G16H 70/60; G16B 40/20; G16B 20/40

(52) U.S. Cl. CPC G16H 50/20; G16H 70/60; G16B 40/20; G16B 20/40

(57) ABSTRACT

Disclosed herein are classifier models, computer implemented systems, machine learning systems and methods thereof for classifying clinical variants of unknown or uncertain significance into a pathogenicity category using measured phenotype features extracted from phenotype assays of transgenic organism expressing the human clinical variant. Embodiments of the present invention relate generally to methods for generating classifier models using machine learning and use of those classifier models to predict the pathogenicity of a clinical variant for a specific human disease (e.g. genetic disease), assigning a patient clinical variant to a pathogenicity category (e.g. pathogenic or benign) for the specific human disease to determine whether that patient should be followed up with additional, more invasive diagnostic testing, or treatment.

### RELATED APPLICATIONS

This application claims priority to U.S. Ser. No. 62/952,219 filed on 20 Dec. 2019 and provisional application U.S. Ser. No. 62/916,141 filed 16 Oct. 2019, each of which are hereby incorporated into this application in their entirety.

### STATEMENT REGARDING FEDERALLY SPONSORED RESEARCH OR DEVELOPMENT

This invention was made with government support under R44GM119906 awarded by the National Institute of General Medical Sciences of the National Institutes of Health. The government has certain rights in the invention.

### FIELD OF THE INVENTION

This application pertains generally to classifier models generated by a machine learning system, trained using phenotypic or transcriptome data from animal models expressing classified clinical variants derived from patients and populations, for predicting pathogenicity of a clinical variant of a disease, especially those clinical variants that are unassigned or of unknown significance.

### BACKGROUND OF THE INVENTION

Clinical genomics is revealing genetic variation occurs at high prevalence in the human population. Accumulated genomic data reveals each person has about 500 sequence variants that create mis sense or indel mutations in the coding regions of their genome (Jansen I et al. Establishing the role of rare coding variants in known Parkinson's disease risk loci. Neurobiol Aging. 2017 November; 59:220.e11-220.e18). With estimates as high as 30% of the genes in the human genome being involved in disease biology (Hegde M et al. Development and Validation of Clinical Whole-Exome and Whole-Genome Sequencing for Detection of Germline Variants in Inherited Disease. Arch Pathol Lab Med. 2017 June; 141(6):798-805.), any one individual harbors over 100 codon-changing variations in their important “disease” genes. Surprisingly, frameshifting indels with a high likelihood of pathogenicity account for only 7% of these variants. As a result, there remains a significant number of questionable alleles that are part of the background of anyone's personal genome. The challenge to the physician is to determine if a suspect allele is contributing to the disease as a pathogenic variant or if the clinical variant is not consequential and can be classified as a benign variant. For many of the genetic differences seen in a patient's genome, the benign or pathogenic status remains undefined and the variant is a Variant of Uncertain Significance (VUS). As a result, variant interpretation is the major bottleneck now that large scale sequencing is increasingly being used in clinical settings.

A significant proportion of clinical variants seen in patients with genetic disease cause missense changes resulting in altered amino acid usage. Unlike the rarer frameshift and stop-codon mutations and some intra-/inter-genic variants, the functional consequence of mis sense amino acid changes can remain elusive. Change of function due to mis sense can result in partial loss of gene activities or gain-of-function changes that are highly pathogenic. There is an emergent need for the functional analysis of variant pathogenicity that occurs as a result of these amino acid changes.

A variety of technologies from bioinformatics to biochemical assays can be deployed to assess functional consequence of mis sense changes. Yet the most reliable are the in vivo systems. Most commonly used are cell culture assays to animal model studies. The lack of intact animal biology occurring cell culture systems renders this technique intractable to many transcellular pathogenicities. As a result, transgenic animal models are favored for capturing the nuances of intra- and inter- cellular pathogenicity in native contexts.

We previously developed methodologies for creating transgenic nematodes and zebrafish using clinical variants, and phenotype assays for evaluating those transgenic nematodes and zebrafish. See U.S. Ser. No. 16/281,988, incorporated herein by reference. As one of the five classical model organisms for genetic studies (worm, fly, yeast, zebrafish and mice) the C. elegans nematode worm has a unique set of attributes that make it highly optimal for high-throughput clinical variant phenotyping. At the genetic level, the C. elegans nematode rivals the Drosophila fly for having orthologs to 80% of human disease genes, wherein 6460 genes detected in ClinVar Miner database as human disease genes were queried for homologs using the DIOPT database (Hu Y et al. An integrative approach to ortholog prediction for disease-focused and other functional studies. BMC Bioinformatics. 2011 Aug. 31; 12:357). Of the multicellular models, the C. elegans animal model has the fastest life cycle (3 days). It has optical transparency for easy tissue and organ system expression observation. Finally, in a unique advantage of interpretability, the C. elegans animals are easy to breed as self-fertilizing hermaphrodites, which allow rapid population expansion of nearly identical animals with very minimal polymorphism load in the genetic background. This allows transgenesis and subsequent population phenotyping to be performed in a matter of a few weeks instead of years.

C. elegans are a microscopic organism, with intact nervous system capable of learned behavior, where the animal can pack into 96 well, 384 well and even 1536 well assays (Leung, C. K., Deonarine, A., Strange, K. & Choe, K. P. High-throughput Screening and Biosensing with Fluorescent C. elegans Strains. J Vis Exp 2011). It has complex tissue structure (nervous system, muscles, germ line, intestine, mouth-like pharynx, periodic excretion through anal sphincter, macrophage-like celomocytes, and a tough skin-like hypodermis). As a result, the C. elegans nematode provides complex tissue biology in an intact, easy-to-culture animal model.

Analysis of genomic data from patient genomic sequences reveals millions of genomic variations occur per person when compared to the reference genome. The challenge to the practicing physician trying to diagnose disease is a need for accurate assessment of given genomic variant's propensity for a dysfunction as a contributor to the disease. Currently, ACMG-AMP guidelines recommend 5 categories of assessment on clinically observed genetic variants: Pathogenic, Likely Pathogenic, Variant of Uncertain Significance, Likely Benign, and Benign. Yet, a large fraction of the variants are deemed to be Variants of Uncertain Significance (VUS). For example, VUS account for 40% of the assignments in the ClinVar database (ncbi.nlm.nih.gov/clinvar). Yet their numbers may be much larger, because current guidelines discourage their inclusion in a clinical report. As a result, clinical reports of genomic sequencing data rely only on a small fraction of variants that have been reliably assessed for pathogenic consequence. As a result, genetic diagnosis of common diseases result in low diagnostic yields. For instance, a recent study performed by GeneDx of patients with epilepsy who had either panel, exome or genome sequencing found the diagnostic rate was only 15% (Lindy A S et al. Diagnostic outcomes for genetic testing of 70 genes in 8565 patients with epilepsy and neurodevelopmental disorders. Epilepsia. 2018 May; 59(5):1062-1071). A significant improvement in diagnostic yield will occur when systems are developed to reliably assess the numerous VUS alleles seen in patient genomic data. Therefore, a need remains for accurately predicting the pathogenicity of VUS alleles for a given disease.

Currently clinical reports rely on performing genomic sequencing on patient DNA to create fastq files. Assembly of fastq against the reference genome creates BAM files. BAM files are queried to identify genetic variations that are collected as an attribute series and then stored as VCF files. Variants are screened utilizing a variety of software tool to annotate the patient genome with variant consequence assessment as per ACMG-AMP (or similar) guidelines. Annotated genomes are refined for salient features into a clinical report that identifies if a variant is causative or a risk factor for disease in question. The present invention overcomes these shortcomings.

It would, therefore, be desirable to provide methods and technologies to permit artificial intelligence/machine learning systems to be used to identify variants of unknown significance as pathogenic or likely pathogenic and to further predict a drug therapy for those patients with that pathogenic or likely pathogenic variant.

Provided herein are classifier models for predicting pathogenicity for a clinical variant of a human disease, wherein the system integrates molecular modeling of variants with in vivo validation in an animal model to increase detection of pathogenicity for VUS.

### SUMMARY OF THE INVENTION

Herein are provided are computer-implemented methods for training a machine learning algorithm to generate a classifier model for predicting pathogenicity for a clinical variant of a human disease and use of that classifier model.

In embodiments provided herein is a computer-implemented method comprising a) obtaining, by one or more processors, a data set comprising measured phenotype and/or transcriptome features of a transgenic organism expressing a human clinical variant, wherein the phenotype and/or transcriptome features are from a population of clinical variants, wherein the clinical variants are labeled with a diagnostic indicator of pathogenic or benign for a specific human disease; b) selecting a subset of the measured phenotype and/or transcriptome features for inputs into a machine learning system, wherein the subset includes at least four phenotype and/or transcriptome features and the diagnostic indicator for the clinical variants; c) randomly partitioning the data set in training data and validation data; and d) generating a classifier model using a machine learning system based on the training data and the subset of inputs, wherein each input has an associated weight, and wherein the classifier model provides binary outcomes selected from pathogenic or likely pathogenic above a pre-determined threshold or benign or likely benign below a pre-determined threshold.

In other embodiments provided herein is a method, in a computer implemented system comprising at least one processor and at least one memory, the at least one memory comprising instructions executed by the at least one processor to cause the at least one processor to implement one or more classifier models to predict pathogenicity for a clinical variant of a human disease, comprising a) obtaining measured phenotype and/or transcriptome features of a transgenic organism expressing the human clinical variant; b) classifying the clinical variant into a pathogenicity category of pathogenic or likely pathogenic using a first classifier model, wherein the first classifier model is generated by a machine learning system using a first training data set that comprises phenotype and/or transcriptome features from the transgenic organism of a panel of at least four phenotype and/or transcriptome features from a population of clinical variants, wherein the clinical variants are labeled with a diagnostic indicator of pathogenic or benign for the human disease, wherein the first classifier model classifies the clinical variant in a pathogenicity category of pathogenic or likely pathogenic using input variables of the measured phenotype and/or transcriptome features of a panel of phenotype and/or transcriptome features from the transgenic organism when an output of the first classifier model is above a predetermined threshold; and, c) providing notification to a user for patient testing when the clinical variant is predicted to be pathogenic or likely pathogenic for a human disease.

### BRIEF DESCRIPTION OF THE DRAWINGS

In the drawings, like numerals describe similar components throughout the several views. Like numerals having different letter suffixes represent different instances of similar components. The drawings illustrate generally, by way of example, but not by way of limitation, various embodiments disclosed herein.

FIG. 1 shows 33 phenotypic features extracted from electropharyngeograms (EPG) recordings of humanized hSTXBP1 nematodes, and the R122X, R406H, V84I, and Y75C hSTXBP1 variant containing nematodes. Each dot represents the recording from one worm.

FIG. 2 shows a table of pathogenic and benign clinical variants of syntaxin binding protein 1 (STXBP1) installed into the nematode and used to train the present classifier model.

FIG. 3 shows 26 phenotypic features extracted from video recordings of humanized hSTXBP1 nematodes, and the D207N, V84I, Y75C, P94L, R122X, R551C, V451I, R406H, P139L, D210N, R406C hSTXBP1 variant containing nematodes. Each dot represents the morphological and motility data from one worm.

FIG. 4 shows data generated from phenotypic features of the transgenic nematodes expressing the clinical variants and the threshold separating benign from pathogenic. Y75C was a clinical variant of unknown significance that was classified using the present classifier model.

FIG. 5 shows the results of holdout analysis of our variant classification. Five known pathogenic variants are listed on the left with the fit quality of their hold out data. Five known benign variants are listed on the right with the fit quality of their hold out data.

FIG. 6 illustrates the F0 Crispinlet knock-in assay, a CRISPR/Cas9-editing F0 lethality screen that detects pathogenicity of clinical variants. FIG. 6A provides generalized schematic of F0 Crispinlet knock-in method. FIG. 6B illustrates the lethality results on STXBP1 with known pathogenic (S42P, R388X and R406H) and known benign (P94L).

FIG. 7 illustrates the F0 Crispin-seq assay, a CRISPR/Cas9-editing F0 Crispin-seq screen detects pathogenicity in clinical variants. FIG. 7A provides generalized schematic of the F0 Crispin-seq method. FIG. 7B shows that high levels of somatic gene conversion lead to high incidence of lethality in known pathogenic STXBP1 variants (S42P, R388X and R406H) while benign variants of STXBP1 (P94L) remain only slightly affected by lethality.

FIG. 8 illustrates a wide diversity of variation in NOTCH3 are associated with pleiotropic disease effects. CADASIL variations predominate in the extracellular domain (A and B) while Lehamans syndrome (E) occurs only with the intracellular domain. Arteriopathy/leukoencephalopathy (C) and Infantile myofibromatosis (D) occur at select positions of extracellular domain. Figure adapted from https://dev.biologists.org/content/144/10/1743.long.

FIG. 9 illustrates the development and assessment of a gene-swap humanized C. elegans animal (FIG. 9a), an exemplary Linear Discriminator Plot (FIG. 9b), an exemplary Receiver Operator Curve (ROC) (FIG. 9c), and exemplary VUS assessment (FIG. 9d).

### DETAILED DESCRIPTION OF THE INVENTION

### Introduction

Embodiments of the present invention relate generally to methods for generating classifier models using machine learning and use of those classifier models to predict the pathogenicity of a clinical variant for a specific human disease (e.g. genetic disease), assigning a patient clinical variant to a pathogenicity category (e.g. pathogenic or benign) for the specific human disease to determine whether that patient should be followed up with additional, more invasive diagnostic testing, or treatment.

Disclosed herein are classifier models and their use with variants of unknown significance from patients as to a specific human disease for prediction of clinical variant pathogenicity, which can inform further patient diagnostic testing or treatment options. The classifier models were generated by a machine learning system using training data that comprises measured phenotype and/or transcriptome features of a transgenic organism expressing a human clinical variant, a diagnostic indicator of the clinical variant, for a population of patient clinical variants. The present classifier models based on measured phenotype features were trained with features extracted from an electropharyngeogram (EPG) assay (e.g., pharyngeal pumping duration, interpump interval, pumping frequency, and peak amplitude of different pump components) or features extracted from an motility and morphology assay (e.g., speed, forward vs. reverse travel, curling, length, width) using transgenic nematodes comprising and expressing the clinical variant. See Example 1, 2 and 6. The present classifier models based on measured transcriptome features can be trained with features extracted from transcriptome data (e.g., RNAseq data). In some embodiments, the classifier models utilize both measured phenotype features and measured transcriptome features.

In embodiments, training data comprises a group of data from a group of clinical variants deemed benign (e.g. the one or more mutations does not lead to disease) and which can include the wild type “wt” allele, and patients with these clinical variants were not diagnosed with that specific disease. In embodiments, the training data comprises a group of data from a group of clinical variants deemed pathogenic (e.g. the one or more mutations did lead to disease) and patients were diagnosed with the specific human disease associated with the allele.

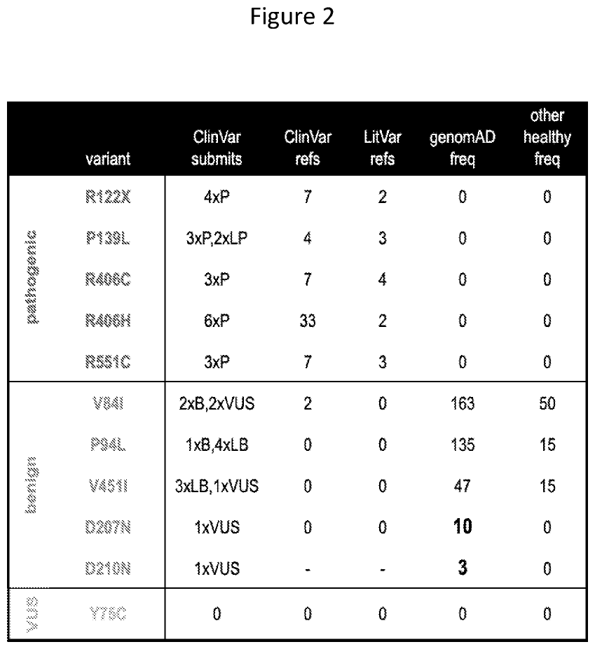

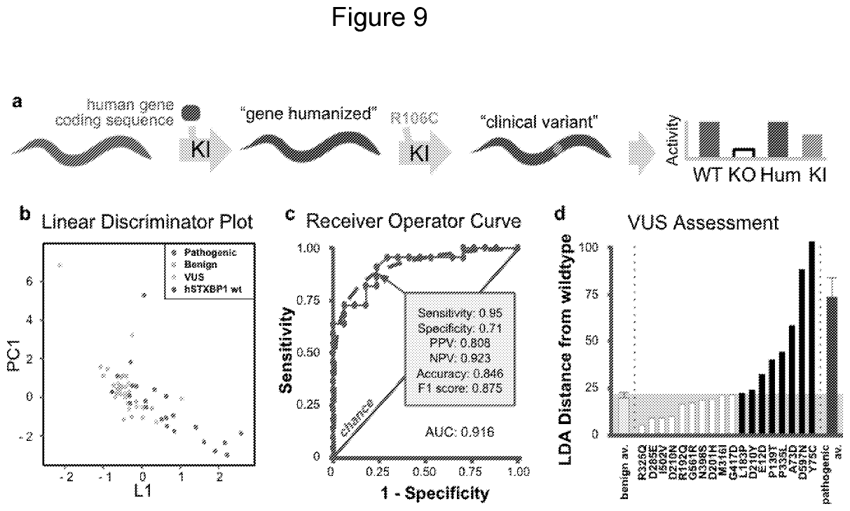

In the present invention, the classifier models are “trained” using machine learning systems by building a model from inputs. Those inputs may be a subset of phenotype and/or transcriptome features of the transgenic organism expressing the clinical variants, wherein the variants were deemed benign or pathogenic based on corresponding patient data (See Clinvar website). See Example 1 for training of the present classifier models using EPG data from transgenic nematodes comprising coding sequences for STXBP1, a protein involved in synaptic vesicle trafficking. Auto somal dominant mutations in STXBP1 are implicated in childhood epilepsies and several neurodevelopmental disorders. See Example 2 for training of the present classifier model using mobility and morphology data from transgenic nematodes comprising coding sequences for STXBP1. See FIG. 2. See Example 6 for training of the present classifier model using phenotypic and morphology data (e.g., wherein the data was harvested in electrophysiology using a screen chip apparatus, and in solid and liquid growth media formats) from transgenic nematodes comprising sequences for STXBP1 and performance of the classifier model as determined with a linear discriminator plot (distance measured from wt) and the corresponding ROC curve demonstrating a sensitivity of 0.95 and specificity of 0.71. See FIG. 9. These data show that a threshold value based on a specificity of 0.70, provides a method to discriminate between benign, or likely benign, and pathogenic, or likely pathogenic, with a sensitivity of about 0.95.

In certain embodiments, the inputs may further comprise clinical variant parameters, such as variant frequency in healthy populations, in silico predictions, human phenotype characterizations from clinical data including related disorder phenotypes, and/or transcriptome features. For example, STXBP1 related disorder phenotypic characterization may include medical terms such as absent speech, cerebral atrophy, cerebral hypomyelination, developmental regression, loss of developmental milestones, EEG with burst suppression, epileptic encephalopathy, epileptic spasms, generalized hypotonia, generalized myoclonic seizures, generalized tonic seizures, generalized tonic-clonic seizures, grand mal seizures, hypoplasia of the corpus callosum, hypsarrhythmia, impaired horizontal smooth pursuit, infantile encephalopathy, intellectual disability, early and severe mental retardation, neonatal onset, severe global developmental delay, spastic paraplegia, spastic tetraplegia, status epilepticus, tremor, and/or variable expressivity.

In embodiments provided herein is a first classifier model, generated by a machine learning system, that classifies a patient clinical variant into a pathogenicity category of pathogenic or benign for a specific human disease (e.g. epilepsy). In embodiments, use of the classifier model classifies a patient clinical variant in a pathogenic category, including likely pathogenic, using input variables of the measured phenotype and/or transcriptome features of a panel of phenotype and/or transcriptome features from the transgenic organism when an output of the classifier model is above a pre-determined threshold value. In other embodiments, the classifier model classifies a patient clinical variant in a benign category using input variables of the measured phenotype and/or transcriptome features of a panel of phenotype and/or transcriptome features from the transgenic organism when an output of the classifier model is below a threshold value. In embodiments, that threshold is determined based on specificity, for example 0.7, or 0.75, or 0.8, or 0.85. In embodiments, the threshold is determined based on the performance of the trained algorithm to optimize the PPV (positive predictive value) and NPV (negative predictive value) so that the algorithm is trained to correctly classify the highest number of true negative (benign) and true positive (pathogenic) variants ensuring the trained classifier model classifies a variant of unknown significance with a sensitivity of at least 90% (0.9).

In certain embodiments the classifier model is static, and its use is implemented by a computer-implemented system comprising at least one processor and at least one memory, the at least one memory comprising instructions executed by the at least one processor to cause the at least one processor to implement the classifier model. In certain embodiments, a machine learning system iteratively regenerates the classifier model by training the classifier model with new training data to improve the performance of the classifier model.

In exemplary embodiments, the present methods using a first classifier model, in a computer implemented system comprising at least one processor and at least one memory, the at least one memory comprising instructions executed by the at least one processor to cause the at least one processor to implement one or more classifier models to predict pathogenicity for a clinical variant of a human disease, comprising obtaining measured phenotype and/or transcriptome features of a transgenic organism expressing the human clinical variant, classifying the clinical variant into a pathogenicity category of pathogenic or likely pathogenic using a first classifier model, wherein the first classifier model is generated by a machine learning system using a first training data set that comprises phenotype and/or transcriptome features from the transgenic organism of a panel of phenotype features (e.g., at least four) and/or transcriptome features from a population of clinical variants, wherein the clinical variants are labeled with a diagnostic indicator of pathogenic or benign for the human disease, wherein the first classifier model classifies the clinical variant in a pathogenicity category of pathogenic or likely pathogenic using input variables of the measured phenotype and/or transcriptome features of a panel of phenotype and/or transcriptome features from the transgenic organism when an output of the first classifier model is above a predetermined threshold and, providing notification to a user for patient testing when the clinical variant is predicted to be pathogenic or likely pathogenic for a human disease.

In some embodiments, variant pathogenicity may be determined by profiling the transcriptome is profiled via RNAseq of CRISPR-edited F0 knock-ins in an assay referred to as “F0 Crisprin-seq”. Zebrafish embryos are injected with CRISPR/Cas9 editing reagents and examined as hatchlings for changes in RNA expression levels as determined using RNAseq data. RNAseq uses next-generation sequencing (NGS) to reveal the presence and quantity of RNA in a biological sample at a given moment, analyzing the continuously changing cellular transcriptome (i.e., the set of all RNA molecules, here specifically mRNA molecules, in one cell or a population of cells). Thus, RNAseq provides a measure of the total expression response of an animal. Applied to variant biology analysis, RNAseq comparison between wild type and a clinical variant can be used to observe changes in expression behavior that are specific to the variant condition. This F0 Crisprin-seq assay system can uncover the quantitative changes in the transcriptome that are specific to the severity of a variant pathogenicity.

In some embodiments, zebrafish can be modified using the “F0 Crispinlet knock-in” system described herein to express a clinical variant gene and assayed to determine, or estimate, the pathogenicity of that clinical variant gene by a phenotypic assay. This method is a CRISPR-based lethality assay in which transgenic zebrafish embryos are modified to express a clinical variant gene and assayed for a phenotypic change (e.g., lethality). In some embodiments, a threshold level of phenotypic change in a population of such transgenic zebrafish indicates the clinical variant is pathogenic. For instance, a pathogenic clinical variant may be one that results in 75% of more lethality (i.e., 25% or less survival) in a population of such transgenic zebrafish. Such methods provide simple, fast and affordable functional testing for detecting pathogenicity in patient-observed clinical variants (i.e., gene variants).

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein “benign” or “likely benign” in the context of a clinical variant, refers to a category of clinical variants that do not comprise one or more disease causing mutations.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

As used herein, “clinical variant” is a disease gene that comprises one or more amino acid changes as compared to wild type and is thus a mutant gene.

As used herein, “clinical variant parameters” refers to additional inputs for the machine learning algorithm, other than “phenotype and/or transcriptome features”, and are generally available from external or other sources, such as medical records or patient records, public databases with information as to specific clinical variants, and published literature. Examples of clinical variant parameters include, but are not limited to, variant frequency in healthy populations, in silico predictions, human phenotype characterizations from clinical data including clinical variant patient related disorder phenotypic characterizations, and/or transcriptome features.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“cDNA” as used herein means the deoxyribonucleic acid sequence that is derived as a copy of a mature messenger RNA sequence and represents the entire coding sequence needed for creation of a fully functional protein sequence.

As used herein, the term “gene editing” refers a type of genetic engineering in which DNA is inserted, replaced, or removed from a genome using gene editing tools. Examples of gene editing tools include, without limitation, zinc finger nucleases, TALEN and CRISPR.

“Genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but is not limited to epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

A “heterologous gene” as used herein refers to a nucleotide sequence not naturally associated with a host animal into which it is introduced, including for example, exon coding sequences from a human gene introduced, as a chimeric heterologous gene, into a host nematode.

As used herein, the terms “increase,” “increased,” “increasing,” “improved,” (and grammatical variations thereof), describe, for example, an increase of at least about 5%, 10%, 15%, 20%, 25%, 35%, 50%, 75%, 80%, 85%, 90%, 95%, 97%), 98%), 99%), or 100% as compared to a control. In embodiments, the increase in the context of a heterogenous gene or clinical variant thereof, is measured and/or determined via a transcriptome assay and/or phenotypic assay to assess function of the expressed gene.

As used herein, the term “genomic locus” or “locus” (plural loci) is the specific location of a gene or DNA sequence on a chromosome and, can include both intron or exon sequences of a particular gene. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, introns, exons, promoter sequences, terminators, translational regulatory sequences such as ribosome binding sites and internal ribosome entry sites, enhancers, silencers, insulators, boundary elements, 5′ or 3′ regulatory sequences, replication origins, matrix attachment sites and locus control regions. As used herein “native locus” refers to the specific location of a host gene (e.g., ortholog to the heterologous gene) in a host animal.

As used herein “machine learning” refers to algorithms that give a computer the ability to learn without being explicitly programmed including algorithms that learn from and make predictions about data. Machine learning algorithms include, but are not limited to, decision tree learning, artificial neural networks (ANN) (also referred to herein as a “neural net”), deep learning neural network, support vector machines, rule base machine learning, random forest, logistic regression, pattern recognition algorithms, etc. For the purposes of clarity, algorithms such as linear regression or logistic regression can be used as part of a machine learning process. However, it is understood that using linear regression or another algorithm as part of a machine learning process is distinct from performing a statistical analysis such as regression with a spreadsheet program such as Excel. The machine learning process has the ability to continually learn and adjust the classifier model as new data becomes available and does not rely on explicit or rules-based programming.

As used herein, the term “medical history” refers to any type of medical information associated with a patient. In some embodiments, the medical history is stored in an electronic medical records database. Medical history may include clinical data (e.g., imaging modalities, blood work, biomarkers, disease samples and control samples, labs, etc.), clinical notes, symptoms, severity of symptoms, family history of a disease, history of illness, treatment and outcomes, an ICD code indicating a particular diagnosis, history of other diseases, radiology reports, imaging studies, reports, medical histories, genetic risk factors identified from genetic testing, genetic mutations, etc.

As used herein, “measured” refers to the detection of a phenotypic feature or a transcriptome feature and assigning a value (e.g., a numerical value) to that feature in a manner that can be used in comparison to another similar feature.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene.

A “normal” or “wild type” nucleic acid, nucleotide sequence, polypeptide or amino acid sequence refers to a naturally occurring or endogenous nucleic acid, nucleotide sequence, polypeptide or amino acid sequence that has not undergone a change. As used herein, the wild type sequence may be a disease gene, but does not comprise a mutation leading to a pathogenic phenotype or altered transcriptome. It is understood there is a distinction between a wild type disease gene (e.g. those without a mutation leading to a pathogenic phenotype and may be an allele reflective of a “normal” heterogenous population) and clinical variants that comprise one or more mutations of those disease genes and that may have a pathogenic phenotype or altered transcriptome. In embodiments, the normal gene or wild type gene may be the most prevalent allele of the gene in a heterogenous population, including wherein the amino acid at each position is the most common found in a heterogeneous population for that allele of the gene such that the “wt” sequence may not appear in anyone individual but is a representative of normal, or non-disease, in the heterogeneous population. In this instance, the wildtype sequence may be a control, and one of the benign variants, used to train the present classifier models and further used to determine the performance of the classifier model. See FIG. 9.

As used herein, the term “pathogenicity category” refers to the status (e.g. clinical significance) of the clinical variant as to a human disease condition. In embodiments, the status includes at least two categories of pathogenic or benign, each of which may include a sub-group of likely pathogenic or likely benign. A source of clinical variant information, including their pathogenicity category and relationship with a human disease condition is the ClinVar website (ncbi.nlm.nih.gov/clinvar). Input variables used to train the present machine learning algorithm used clinical variants with pre-determined pathogenicity category and the associated human disease condition phenotype and/or transcriptome (i.e. diagnostic indicator).

As used herein “pathogenic” or “likely pathogenic” in the context of a clinical variant, refers to a category of clinical variants that comprise one or more disease causing mutations.

“Partially-functional” as used herein describes a protein that is encoded by a mutant gene and has less biological activity than a functional protein but more than a non-functional protein. In embodiments, function is determined via one or more phenotypic assays wherein a phenotypic profile for the mutant (disease) gene may be generated.

As used herein “phenotype feature”, “transcriptome feature” or “phenotype and/or transcriptome features” refers to inputs for the machine learning algorithm that are measured and extracted from phenotype or transcriptome assays using the transgenic organism expressing a human clinical variant. Examples of phenotype features extracted from an EPG assay include, but are not limited to, pharyngeal pumping duration, interpump interval, pumping frequency, or peak amplitude of different pump components. Examples of phenotype features extracted from motility or morphology assay include, but are not limited to, speed, forward vs. reverse travel, curling, length, or width. Examples of transcriptome features extracted from a gene expression, specifically a transcriptome assay such as an RNA expression assay, and more specifically an mRNA expression assay including, but not limited to, an RNAseq assay. RNAseq uses RNA sequencing, e.g., next-generation sequencing (NGS), to reveal the presence and quantity of RNA in a biological sample at a given moment, analyzing the continuously changing cellular or organismal transcriptome (i.e., the set of all RNA molecules in one cell or a population of cells or organisms) (see, e.g., Chu, et al. Nuc. Acids Ther., 22(4): 271-4 (2012) and Wang, et al. Nature Rev. Genetics, 10(1): 57-63 (2009). In some embodiments, the RNA can be total RNA, any particular type of RNA (e.g., small RNA, miRNA, tRNA, ribosomal RNA), and/or messenger RNA (mRNA), and/or fragments thereof. In preferred embodiments, the RNA measured is mRNA. Examples of transcriptome features extracted from a RNAseq assay include, but are not limited to, transcriptome profiles which reveals the types and/or amounts of RNA, e.g., mRNA, expressed in a cell, tissue, and/or organism.

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%), 98%), 99%), or 100% as compared to a control. In embodiments, the reduction in the context of a heterogenous gene or clinical variant thereof, is measured and/or determined via phenotypic assay to assess function of the expressed gene, and/or via a transcriptome assay.

As used herein the term, “Receiver Operating Characteristic Curve,” or, “ROC curve,” is a plot of the performance of a particular model for distinguishing two populations, pathogenic clinical variants, and controls, i.e., clinical variants from patients with no explicit diagnosis of disease (e.g. benign clinical variants). Data across the entire population (namely, the pathogenic clinical variants and controls) are sorted in ascending order based on the value of a single feature or composite of features. Then, for each value for that feature, the true positive and false positive rates for the data are determined. The true positive rate is determined by counting the number of cases above the value for that feature under consideration and then dividing by the total number pathogenic clinical variants. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied, weighted, etc.) to provide a single combined value which can be plotted in a ROC curve. The ROC curve is a plot of the true positive rate (sensitivity) of a test against the false positive rate (1-specificity) of the test. ROC curves provide another means to quickly screen a data set. As used herein, performance of the present classifier models may be determined using computed ROC curves with sensitivity and specificity values. The performance is used to compare models, and also importantly, to compare models with different variables to select a classifier model with the highest accuracy as to predicting clinical variants as likely pathogenic, for a patient.

“Subject” and “patient” as used herein interchangeably refers to any vertebrate, including, but is not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. The subject or patient may be undergoing other forms of treatment. In embodiments, the patient is a human wherein a clinical variant is a sequence of a disease gene from the patient.

“Target gene” as used herein refers to any nucleotide sequence encoding a known or putative gene product. As used herein the target gene may be the chimeric heterologous gene, either in normal or wild type form, or as a clinical variant, or the host animal ortholog of the heterologous gene. The target gene may be a mutated gene involved in a genetic disease, also referred to herein as a clinical variant.

“Variant” with respect to a peptide or polypeptide that differs in one or more amino acid sequence by the insertion, deletion, or conservative substitution of amino acids as compared to a normal or wild type sequence. The variant may further exhibit (or induce) a phenotype and/or transcriptome that is quantitatively distinguished from a phenotype of the normal or wild type expressed gene and/or transcriptome. In embodiments, clinical variant refers to a disease gene with one or more amino acid changes as compared to the normal or wild type disease gene.

### Classifier Models Generated by Machine Learning Systems and Their Use

Disclosed herein are classifier models, computer implemented systems, machine learning systems and methods thereof for classifying patient clinical variants of unknown significance (i.e. variants that have not been deemed benign or pathogenic, status is unknown or uncertain) into a pathogenicity category of pathogenic or likely pathogenic (or benign or likely benign) for a specific human disease.

The present classifier models are generated using machine learning algorithms with phenotype and/or transcriptome features as inputs wherein those features are measured in a phenotype or transcriptome assay using a transgenic organism expressing the clinical variants. In embodiments, the training data set used to generate the classifier model comprises patient clinical variants deemed pathogenic or benign for a specific human disease. For simplicity, benign clinical variants may also be referred to herein as a “control” or “negative” sample. In embodiments, the present classifier models are generated using clinical variants of the same allele, which correspond to a specific human disease. In embodiments, the allele or specific disease are used to select the classifier model. In embodiments, in the situation where training data comprises a greater number of one class of variants than the other class, training of the classifier models comprises reprocessing the training data by using a stratified sampling technique to preserve the relative proportion of the two class examples in the training data as in the validation data.

In embodiments, the machine learning system generates a classifier model that may be static. In other words, the classifier model is trained and then its use is implemented with a computer implemented system wherein clinical variant data (e.g., measure phenotype and/or transcriptome features of the transgenic organism expressing the clinical variant) are input and the classifier model provides an output that is used to classify patient clinical variants. In other embodiments, the classifier models are continuously, or routinely, being updated and improved wherein the input values, output values, along with a diagnostic indicator from patients are used to further train the classifier models.

In embodiments, the first classifier model classifies the patient clinical variant in a pathogenic category using input variables of measured phenotype and/or transcriptome features of the transgenic organism expressing the clinical variant when an output of the first classifier model is above a pre-determined threshold. In embodiments, the first classifier model classifies the patient clinical variant in a benign category using input variables of measured phenotype and/or transcriptome features of the transgenic organism expressing the clinical variant when an output of the first classifier model is below a pre-determined threshold. In certain embodiments, the output is a probability value, wherein the threshold is set to separate patient clinical variants into a benign category from a pathogenic category. In certain embodiments, the pathogenic category may be further subdivided, such as a likely pathogenic category and a pathogenic category. In certain embodiments, the pathogenic category may be further subdivided into different categories based on pathological mechanism. In embodiments, the inputs for the classifier model further comprises clinical variant parameters.

Aspects of embodiments of the present invention are inextricably tied to computing at least because the electronic models, including automatically generated self-learning predictive models generated from training data, generated by embodiments of the present invention cannot be generated outside of computing and do not exist outside of computing. Records initially utilized in embodiments of the present invention are electronic records in one or more data set, contained in one or more database, that are machine readable. The resultant models are also electronic and are applied to additional electronic data sets utilizing computing resources. Because of both the volume and the nature of data, an individual is not capable of accomplishing the specific aspects of embodiments of the present invention that result in a machine readable data model that can be applied by program code to additional data sets in order to identify records with a probability of an event or condition that the model was generated to predict the probable pathogenicity of a clinical variant for a specific human disease.

Embodiments of the present invention provide advantages and improvements that are inextricably tied to computer technology also because embodiments of the present invention offer certain advantages that increase computational efficiency and efficacy. For example, embodiments of the present invention utilize distributed processing based on anticipated query results in order to decrease the timeline for key analytic deliverables. This distributed processing enables the program code to perform multiple analysis processes simultaneously. Portions of certain embodiments of the present invention can be migrated to a cloud architecture and made available to users as software as a service (SaaS) offerings. The unlimited computational capacity of resources in a cloud architecture are suited to support the program code's distribution of simultaneous queries and processes in order to meet the efficiency demands of the system in a data rich environment.

In an embodiment of the present invention, one or more programs exploit principal component analysis (PCA), which is a statistical procedure, to determine a related set of concepts or components to one or more features matching a number of individuals (e.g. transgenic nematode expressing clinical variant) in a training set of data. PCA comprises an orthogonal transformation to convert a set of observations of possibly correlated variables into a set of values of linearly uncorrelated variables called principal components. The number of principal components is less than or equal to the number of original variables. Thus, in an embodiment of the present invention, the program code exploits a parent concept to generate multiple sub-components. This transformation is defined in such a way that the first principal component has the largest possible variance (that is, accounts for as much of the variability in the data as possible), and each succeeding component in turn has the highest variance possible under the constraint that it is orthogonal to the preceding components. The resulting vectors are an uncorrelated orthogonal basis set. PCA is sensitive to the relative scaling of the original variables.

In an embodiment of the present invention, the raw electronic data comprises raw data from one or more phenotype and/or transcriptome assays, wherein the assay records one or more features. The data set may include one to 100 values, where each clinical variant represents one value and multiple data sets represent the one value. The data set includes data pertaining to clinical variants that were previously categorized as pathogenic (patient was diagnosed with a specific disease) or benign (patients who do not have that diagnosis); the diagnostic indicator of the clinical variant. The records in the data set are labeled to indicate this characterization.

In embodiments, the features are selected from pharyngeal pumping duration, inter-pump interval, pumping frequency, peak amplitude of different pump components, speed, forward vs. reverse travel, curling, length, width, lethality, attenuation, bending angle-mid-point asymmetry, maximum amplitude (um), self-contact distance, mean amplitude (um), body wave number, area, dynamic amplitude (stretch), center point speed (um/s), center point trajectory/time, peristaltic speed (um/s), absolute peristaltic track length/time, activity, brush stroke, length, reverse swim, curling, fit, swimming speed, wave initiation rate, wavelength, width, proportion time forward, proportion time reverse, straight-line speed, forward speed, and reverse speed.

Utilizing the data set, the one or more programs generate a predictive model that distinguishes the data related to individuals with pathogenic clinical variant from individuals represented by the data with a benign clinical variant. The predictive model generated by the one or more programs in embodiments of the present invention can be understood as a classifier or a classifier algorithm. The one or more programs, in generating the predictive model, utilizing the data set as training data, and generates, based on the training provided by this data set, the classifier model.

In an embodiment of the present invention, the one or more programs apply the classifier model to the data related to a clinical variant of unknown significance from an individual to determine whether the data related to the individual indicate a classification as pathogenic (or likely pathogenic) for a specific human disease (or condition/syndrome). The condition may be a genetic disease or condition. In embodiments, the disease is selected from epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, or Tay-Sachs disease. In other embodiments, the disease may be cancer, a neurodegenerative disease or metabolic disease.

In embodiments, cross-validation is used to train and evaluate the classifier model performance. To perform a k-fold cross validation, a training dataset is divided into k groups in a stratified manner. In k iterations, each group is designated as the hold-out fold and the remaining k-1 groups are the training set. Each of the k groups will be designated as the hold-out set exactly once. During each iteration, the classifier model is trained on k-1 groups (training set) and accuracy evaluated on the one hold-out fold.

In certain embodiments, the one or more programs process data in order to improve the efficiency of the model generation. In some embodiments of the present invention, the one or more programs separate the data into an amino acid sequence part and feature part (e.g., measured phenotype and/or transcriptome features). The one or more programs separate the records into a pre-defined number of groups, by assigning records to groups randomly. In some embodiments of the present invention, the one or more programs generate multiple random seeds and for each seed, separate the records into the pre-defined number of groups. For example, if a random number is six (6), the one or more programs will assign each 6th record to one of the groups. The one or more programs will continually generate various seeds and utilize these seeds to make the random assignments of the records to the groups. The groups are generated by the one or more programs such as each group contains an equal (or similar) amount of data as each other group.

In certain embodiments, the one or more programs execute a PCA to determine features that are relevant to clinical variants represented in the test data. In the PCA, the one or more programs determine which features are most representative in the test data. In part as a result of the PCA, the eventual model generated by the one or more programs may include a cumulative understanding of the features common to the individuals. The one or more programs utilize this data to infer which features have the highest variance and the one or more programs may determine an order for the terms.

In embodiments, for every step of cross validation, the one or more programs may run PCA on the training set and transform the validation using the loadings from this PCA result, before feeding them into the model for prediction, to compute validation accuracy. In certain embodiments, the one or more programs perform a PCA on each training set, per cross validation step, as well as the final evaluation step, and write the list of results to a file for each sub data set. The one or more programs read this PCA result file will be read into memory when its contents are utilized in cross validation and modeling.

In certain embodiments, the one or more programs generate a predictive model by utilizing a number of features identified through PCA (e.g., selected based on dominance) as a parameter for a best fit in a Logistic Regression (LR) model, Linear discriminant analysis (LDA), Support vector machine with linear kernel (Linear SVM) or other linear classification algorithm model, in order to obtain a (predicted) binary outcome (e.g., pathogenic or benign). In certain other embodiments, a non-linear classification algorithm, such as support vector machine with radial basis function kernel, is used to generate the predictive model. In certain embodiments, the one or more programs apply a PCA to the data set and utilize the resultant principal components to build a linear classification model, with the aim to predict (binary) class labels, in this case, pathogenic or benign. In certain embodiments, the one or more programs utilize cross validation to tune the best number of principal components to be included in the model (e.g., 10 means the first 10 components, 100 means the first 100 components). The one or more programs select the smallest (best) number that yields the highest validation for the number of folds. For example, in embodiments of the present invention that utilize a total of six groups and 5-fold validation, the one or more programs select the smallest (best) number that yields the highest 5-fold validation accuracy. The one or more programs evaluate the accuracy of the test based on a model using this parameter. In these 6-group embodiments of the present invention, the one or more programs perform the cross-validation and determine that a linear classification model with several principal components from the measured phenotype and/or transcriptome features provide an average validation accuracy up to 95%.

In exemplary embodiments, linear discriminant analysis (LDA) achieved a recall score of 0.849, a precision score of 0.849, and an f1 score of 0.848; support vector machine with linear kernel (Linear SVM), achieved a recall score of 0.852, a precision score of 0.853, and an f1 score of 0.852; and, support vector machine with radial basis function kernel, achieved a recall score of 0.830, a precision score of 0.843, and an f1 score of 0.830. See Example 1. In certain embodiments, the one or more programs select the features that yields a highest validation accuracy and validation AUC. See Example 6.

In embodiments, the one or more programs obtain a new sample of data comprising a variant of unknown significance expressed in a transgenic organism and the measured phenotype and/or transcriptome features. The one or more programs score the clinical variant for classifying as pathogenic or benign for a specific human disease with a probability by applying the tuned predictive (classifier) model. The one or more programs apply the tuned predictive model to generate the (binary) prediction (e.g., probability of pathogenic or benign, based on the model).

In embodiments, the first classifier model comprises a support vector machine, a decision tree, a random forest, a neural network, a deep learning neural network, or a logistic regression algorithm. In other embodiments, the classifier model comprises a pattern recognition algorithm. In certain embodiments, the classifier model comprises k-Nearest Neighbors algorithm (kNN).

Disclosed herein is a machine learning system comprising at least one processor for predicting classification of a clinical variant of unknown significance from an individual patient as pathogenic or benign for a specific disease.

In certain embodiments, the processor is configured to obtain measured phenotype and/or transcriptome features of a transgenic organism expressing the human clinical variant; classify the clinical variant into a pathogenicity category of pathogenic or likely pathogenic using a first classifier model, wherein the first classifier model is generated by a machine learning system using a first training data set that comprises phenotype and/or transcriptome features from the transgenic organism of a panel of at least four phenotype and/or transcriptome features from a population of clinical variants, wherein the clinical variants are labeled with a diagnostic indicator of pathogenic or benign for the human disease, wherein the first classifier model classifies the clinical variant in a pathogenicity category of pathogenic or likely pathogenic using input variables of the measured phenotype and/or transcriptome features of a panel of phenotype and/or transcriptome features from the transgenic organism when an output of the first classifier model is above a predetermined threshold; and, providing notification to a user for patient testing when the clinical variant is predicted to be pathogenic or likely pathogenic for a human disease.

### Transgenic Organism

In embodiments, the transgenic organism may be any organism that expresses the human clinical variant and configured for use in a phenotype assay or transcriptome assay wherein more than one feature is measured. In embodiments, the transgenic organism is a nematode or zebrafish. In exemplary embodiments, the transgenic organism is a nematode wherein the human gene replaces the nematode ortholog at the native locus. In exemplary embodiments, the transgenic organism is a zebrafish wherein the human gene replaces the zebrafish ortholog at the native locus. In some embodiments, a human gene can be inserted at a safe harbor locus in zebrafish, establishing the human gene can rescue function of a null using the ortholog promoter or some other promoter, and then variants can be installed into the zebrafish genome followed by measurement of phenotypic consequence(s).

Exemplary embodiments provided herein include a transgenic animal system wherein an entire host animal ortholog is replaced with a chimeric heterologous gene, wherein the heterologous gene rescues (or at least partially restores) function of the removed animal ortholog. As used herein, this method of replacing the host animal ortholog with the chimeric heterologous gene, may also be referenced as “gene-swap”. As used herein, “chimeric heterologous gene” refers to a sequence comprising heterologous (to the host animal) exon coding sequences interspersed, or paired, with artificial (or modified) host animal intron sequences, wherein the chimeric heterologous gene is optimized for expression in the host animal which may include codon optimization and removal of any aberrant splice donor and/or acceptor sites that were introduced as a function of the chimeric sequences. In embodiments, the heterologous exon coding sequences are “wild type” or from an allele that is reflective of a heterogenous population. In certain embodiments, the heterologous exon coding sequences are from human genes (e.g. clinical variants).

In embodiments, the transgenic organism expresses a clinical variant, which when expressed comprise one or more amino acid changes as compared to the wild type heterologous gene, is installed in the heterologous gene via site directed mutagenesis. Clinical variants are typically classified as pathogenic, likely pathogenic, benign, likely benign or a variant of unknown significance (VUS). The system provides a platform that can be used to test the function of those variants of the heterologous genes (e.g. human clinical variants) and provided as phenotype and/or transcriptome features (e.g. inputs for the classifier models).

The animals of the invention are “genetically modified” or “transgenic,” which means that they have a transgene, or other foreign DNA, added or incorporated, or an endogenous gene modified, including, targeted, recombined, interrupted, deleted, disrupted, replaced, suppressed, enhanced, or otherwise altered, to mediate a genotypic (e.g., transcriptome) or phenotypic effect in at least one cell of the animal and typically into at least one germ line cell of the animal. In some embodiments, the animal may have the transgene integrated on one allele of its genome (heterozygous transgenic). In other embodiments, animal may have the transgene on two alleles (homozygous transgenic). In some embodiments, the animal may be transiently expressing the protein.

In certain embodiments, the transgenic animals are model organisms including, but not limited to, nematodes, zebrafish, fruit fly, xenopus, or rodents, such as mice and rats.

In certain embodiments, the present transgenic animals provide a single gene copy wherein a chimeric optimized cDNA of a heterologous gene, e.g. modified human cDNA, is inserted to replace coding sequences of a C. elegans ortholog. The humanized animal is then compared to an animal lacking that C. elegans gene, to confirm significant restoration of wild type function. The validated transgenic animal is then modified by installation of a clinical variant and tested in one or more phenotyping assays to measure phenotype features or transcriptome assays to measure transcriptome features of the transgenic organism expressing the human clinical variant. These transgenic animal models have distinct advantages for testing, exploring variant biology and classifying variants or unknown significance as pathogenic or benign using the present classifier models. For example, humanized models circumvent differences in compound binding between humans and other species. Moreover, preliminary results show that gene-swapped loci may be more sensitive to pathogenic variant activity, as compared to pathogenic variant installation in the C. elegans gene.

In embodiments, the chimeric heterologous gene comprises human heterologous exon coding sequences interspersed, or paired, with artificial host nematode intron sequences optimized for expression in the host nematode. In embodiments, the host nematode intron coding sequences are from a highly expressed C. elegans gene and may be further modified for optimized expression.

The instant transgenic nematode system comprises a host nematode that comprises a chimeric heterologous gene, wherein the entire host nematode ortholog was removed, either prior to or at the same time the chimeric heterologous gene was installed, and wherein the chimeric heterologous gene is installed at the host nematode ortholog native locus. In embodiments, the host nematode is a C. elegans, C. briggsae, C remanei, C. tropicalis, or P. pacificus. (Sugi T et al. Genome Editing in C. elegans and Other Nematode Species. Int J Mol Sci. 2016 Feb. 26; 17(3):295).

In some embodiments, a transgenic zebrafish system comprises a host zebrafish that comprises a chimeric heterologous gene, wherein the entire host zebrafish ortholog was removed, either prior to or at the same time the chimeric heterologous gene was installed, and wherein the chimeric heterologous gene is installed at the host zebrafish ortholog native locus. In some embodiments, the chimeric heterologous gene comprises human heterologous exon coding sequences interspersed, or paired, with artificial host zebrafish intron sequences optimized for expression in the host zebrafish. In embodiments, the host zebrafish intron coding sequences may be further modified for optimized expression. In some embodiments, a transgenic zebrafish system can comprise a host zebrafish wherein the chimeric heterolog gene is installed at the native locus coding sequence and contains at least one nucleotide change creating the missense, nonsense, frameshift or splice variation (i.e., variant) that is seen in the patient.

In embodiments, the heterologous gene is a human gene. In embodiments, the chimeric heterologous gene replaces the entire nematode ortholog gene at the native locus, accordingly the chimeric heterologous gene must have a homolog as an identified ortholog in the host nematode. In one embodiment, the homolog is of substantial quality when sequence identity between heterolog source and host exceeds 70%. In one embodiment, the homolog is of high quality when sequence identity between heterolog source and host exceeds 50%. In other embodiments, the homolog is good when its identity exceeds 35%. In other embodiments, the homolog is adequate when its identity exceeds 20%. In other embodiments, the homolog is poor but acceptable when its identity is less than 20%.

In embodiments, the heterologous gene is a human disease gene. As used herein, “disease gene” refers to a gene involved in or implicated in a disease. In certain embodiments provided herein are transgenic nematodes comprising a heterologous gene that is a human wild type disease gene that has replaced the host nematode ortholog at the native locus. In embodiments, the chimeric heterologous gene rescues, or at least partially restores, function of the removed host nematode ortholog. Rescue or restoration of function, which is measured in a phenotypic assay or transcriptome assay, identifies those transgenic nematodes that are validated and may be used as a transgenic control animal. As used herein “validated transgenic control nematode” means a transgenic nematode expressing a chimeric heterologous gene in place of the host nematode ortholog, wherein at least partial function is rescued by expression of the heterologous gene. Rescued function can be from 1% to 100% as compared to wild type host nematode, referred to in the examples and figures as N2. As used herein “validated transgenic control zebrafish” means a transgenic zebrafish expressing a chimeric heterologous gene in place of the host zebrafish ortholog, or a gene edited native locus (e.g., using CRISPR), wherein at least partial function is rescued by expression of the heterologous or edited gene. Rescued function can be from 1% to 100% as compared to wild type host zebrafish.

In addition to quantitative rescue effects, rescue can be qualitative as to essential genes, wherein rescue with a heterologous transgene provides sufficient lifespan and fecundity for establishment of a propagating colony.

In embodiments, rescue of function is measured by analyzing, observing or monitoring the transgenic nematodes in a phenotypic assay as compared to wild type host nematodes and/or null variants. In embodiments, the phenotypic assay is selected from a measurement of electrophysiology of pharynx pumping, a food race, lifespan assay, extension and contraction assay, movement assay, fecundity assay with egg lay or population expansion, apoptotic body formation, chemotaxis, lipid metabolism assay, body morphology changes, fluorescence changes, drug sensitivity and resistance assays, or a combination thereof. There is no limitation as to the phenotypic assay that may be used, including those developed in the future, provided a useful phenotype profile can be generated for assessing function of the installed chimeric heterologous gene. The above are representative phenotype assays, but others may be used to validate the transgenic nematode, as well as for assessing variants of the heterologous genes.

In embodiments, a phenotype profile of the transgenic nematode is identified from the assay wherein the identified phenotype is selected from electropharyngeogram variant, feeding behavior variant, defecation behavior variant, lifespan variant, electrotaxis variant, chemotaxis variant, thermotaxis variant, mechanosensation variant, movement variant, locomotion variant, pigmentation variant, embryonic development variant, organ system morphology variant, metabolism variant, fertility variant, dauer formation variant, stress response variant, or a combination thereof.

In embodiments, the present validated transgenic nematodes are prepared via homologous recombination at the native locus of the host nematode ortholog wherein the nematode ortholog is replaced with the heterologous gene. This method is advantageous in that it provides a platform for further testing and modifications and provides an improvement over previously disclosed methods that use amino acid substitution for generation of humanized nematodes expressing clinical variants. The use of gene-swap (i.e. heterologous gene replaces the nematode ortholog at the native locus) avoids the expression level issues that are a challenging problem with extrachromosomal array studies. Instead, CRISPR techniques are deployed to directly mutate at native loci. Farboud B and Meyer B J. Dramatic enhancement of genome editing by CRISPR/Cas9 through improved guide RNA design. Genetics. 2015 April; 199(4):959-71; Paix A et al. High Efficiency, Homology-Directed Genome Editing in Caenorhabditis elegans Using CRISPR-Cas9 Ribonucleoprotein Complexes. Genetics. 2015 September; 201(1):47-54.

Gene swap involves removal of the native coding sequence of the host nematode (e.g. C. elegans) ortholog and replacement with cDNA from the heterologous gene (e.g., human gene), wherein the exon coding sequences of the heterologous gene are paired with, or interspersed with, host nematode intron sequences. The host intron sequences are derived from a highly expressed host gene and may be further modified for expression of the heterologous exon coding sequences. As used herein “chimeric heterologous gene” refers to a sequence of heterologous (to the host animal) exon coding sequences that are paired or interspersed with the host animal intron sequences.

To execute a gene-swap, the coding sequence from heterologous cDNA is optionally adjusted for optimal expression in the host nematode, e.g., C. elegans, or host zebrafish. In addition to the use of host animal intron sequences paired with heterologous exon coding sequences, optimization includes codon optimization for the host animal and removal of any aberrant splice donor and/or acceptor sites that were generated as a result of the chimeric sequence. Accordingly, in embodiments provided herein are transgenic nematodes comprising a chimeric heterologous gene optimized for expression in the host nematode wherein the heterologous gene replaced a host nematode or zebrafish gene ortholog, wherein the chimeric heterologous gene comprises heterologous exon coding sequences interspersed with artificial host nematode or zebrafish intron sequences.

In embodiments, optimization comprises codon optimization (e.g. removal of rare codons), introduction of host intron sequences into the heterologous cDNA and removal of any aberrant splice sites. For codon optimization, rare codon usage must be avoided to enable sufficient levels of protein translation from a mRNA message. For intron sequences, the artificial host intron sequences are added to the codon optimized heterologous cDNA sequence, which results in improved mRNA stability, and a chimeric sequence. Performing those techniques are well known in the art and online tools exist for performing both. Conveniently, codon optimization and identification of aberrant splice sites are achieved with the C elegans codon adapter that encodes optimal amino acid sequence (Redemann S et al., C. elegans codon Adapter—GGA, Nat Methods. 2011 March; 8(3):250-2) and NextGene2 which adjust splice donor and acceptor sites for optimal performance (Hebesgaard S M et al., Nucleic Acids Res. 1996 Sep. 1; 24(17):3439-52).

Those chimeric sequences, heterologous cDNA optimized, and artificial host intron sequences added may result in a sequence with highly repetitive sequences that prevent gene synthesis by DNA sequence providers. As a result, the sequence may be hand curated to minimize repeat sequence formation and enable synthesis to proceed from suppliers. The need to hand curate sequence content creates a need for removal of aberrant splice site donor and acceptor site. Online tools exist for identify unintentional splice site donor and acceptor sites. Additional hand curated sequence adjustments are made iteratively until on-line software no longer detects aberrant splice site donor and acceptor sites.

In certain embodiments, the transgenic control nematodes or zebrafish may be prepared by methods other than homologous recombination into the native locus, provided the cDNA of the heterologous gene is optimized for expression in the host nematode by codon optimization, addition of host intron sequences to the cDNA sequence of the heterologous gene and removing aberrant splice donor and acceptor sites. Those alternative methods comprise inserting the optimized chimeric heterologous gene via homologous recombination into a native locus of the nematode wherein a nematode or zebrafish gene ortholog is removed, wherein the heterologous gene rescued, or at least partially restored, function of the removed nematode or zebrafish ortholog; or, inserting the optimized heterologous gene into a non-native locus of the nematode or zebrafish; or, inserting the optimized heterologous gene into a random site of the nematode or zebrafish genome; or, adding the optimized heterologous gene as an expression vector wherein the optimized heterologous gene is not integrated into the nematode or zebrafish genome.

In embodiments are provided transgenic test nematodes or zebrafish, which are based on the validated transgenic control nematode and comprise a clinical variant of the heterologous gene. As used herein, “variant heterologous gene” refers to an expressed gene with one or more amino acid changes as compared to the heterologous gene that was used to prepare the validated transgenic control nematode or zebrafish. In embodiments, the variant heterologous gene is a human clinical variant. Accordingly, a transgenic test nematode or zebrafish comprises a transgenic control nematode or zebrafish that is a modified validated transgenic nematode or zebrafish, wherein the expressed heterologous gene comprises one or more amino acid changes providing a variant of the heterologous gene. The transgenic test nematodes or zebrafish, expressing a clinical variant, are used in phenotype and/or transcriptome assays to generate input values for the present classifier models. In embodiments, a transgenic test nematode or zebrafish comprises a chimeric variant heterologous gene, comprising heterologous exon coding sequences interspersed with artificial host nematode intron sequences optimized for expression in the host nematode or zebrafish, wherein the exon coding sequences comprise one or more mutations resulting in an amino acid change as compared to a wildtype reference sequence (wild type heterologous gene of transgenic control animal), and wherein the chimeric variant heterologous gene replaced an entire host nematode or zebrafish gene ortholog at a native locus, and wherein the heterologous gene is a eukaryotic gene.

In embodiments, the variant heterologous gene may be introduced by amino acid swap of the transgenic control nematode or zebrafish or gene swap of a variant containing heterologous gene in as replacement of the unc-18 coding sequence. In embodiments, the variant heterologous gene is a human disease gene comprising one or more amino acid changes as compared to the wild type disease gene. In embodiments, the clinical variant comprises a single amino acid change wherein the change was installed into the integrated heterologous sequence of the transgenic control animal via a co-CRIPSR method. In certain embodiments, the mutations (of the heterologous exon coding sequence) are created from a pool of DNA repair templates each containing one or more mutations. In other embodiments, the clinical variant comprises more than one amino acid change. In certain embodiments, those mutations are created from a pool of DNA repair templates each containing two or more mutations. Clinical variants with more than one amino acid change, as compared to the wild type gene, may be a known clinical variant or a combination of two or more variants of the same gene.

In embodiments, the variant heterologous gene is a human clinical variant. Accordingly, when at least partial rescue of function is achieved with expression of the heterologous gene, the system (comprising validated transgenic nematodes) becomes valid for installation of clinical variants (test transgenic nematodes). Six classes of clinical variants can be installed (Pathogenic, Likely Pathogenic, Uncertain Significance, Likely Benign, Benign, and the unassessed). On average, dbSNP data indicates 80% of known variants are unassessed and nearly half (40%) of the remaining assessed variants are Variants of Uncertain Significance (VUS). (NCBI) Variation Viewer. Installation of known Pathogenic and Benign variants to prepare transgenic nematodes or zebrafish were used to generate the training and validation data sets to train the preset classifier models.

In embodiments, clinically relevant point mutations in genes are modeled in animals. In certain embodiments, C. elegans models are created using CRISPR/Cas9 or by other genome editing techniques such as MosSCI or extrachromosomal arrays. Zebrafish models are created by CRISPR/Cas9 site-directed mutagenesis of native orthologous genes. Zebrafish models can be inherited germline edits of the native chromosome or somatic CRISPR-mediated mutants (crispants). Alternatively, knock-down (morpholino) or knock-out lines can be rescued by human mRNA or expression plasmids to create animals expressing clinically relevant proteins.

In embodiments, methods are provided herein for assessing function of a human clinical variant, comprising the steps of culturing (e.g., growing) a test transgenic animal (e.g., nematode or zebrafish), wherein the variant heterologous gene is a human clinical variant; and, performing a phenotypic or transcriptomic screen to identify a phenotype and/or transcriptome of the test transgenic animal (e.g., nematode or zebrafish), wherein a change in phenotype or transcriptome as compared to a control transgenic animal comprising a wildtype heterologous gene (e.g. corresponding validated transgenic animal) indicates an altered function of the clinical variant in the test transgenic animal (e.g., any statistically significant deviation from control which is typically the wild type gene but can also be a knock-out). The phenotypic screens and identified phenotypes, and/or transcriptomic screens and identified transcriptomes, are disclosed above and are the same as those used when validating the transgenic control animal for rescue of function. From those phenotype and/or transcriptome assays features are extracted and used to generate the classifier models. See Example 1.

Animal models are characterized using multiple phenotyping or transcriptome assays. Behavioral, survival, developmental, morphological, and molecular phenotyping assays are used. Behavioral assays include: locomotion, chemosensation, mechanosensation, osmotic avoidance, thermal response, feeding, reproduction, learning, swimming, egg-laying, defecation, galvanotaxis, and circadian rhythms. (Hart, Anne C., ed. Behavior (Jul. 3, 2006), WormBook, ed. The C. elegans Research Community, WormBook, doi/10.1895/wormbook.1.87.1, http://www.wormbook.org.) Survival assays include; abiotic stress resistance (oxidative, hypoxic, hyperoxic, heat, cold, heavy metal, ER, UV, osmotic, etc.), pathogen resistance (gram-positive pathogenic bacteria, gram-negative pathogenic bacteria, fungi, parasites, etc.) and healthspan/lifespan (Park H H, Jung Y, Lee S V. Survival assays using Caenorhabditis elegans. Mol Cells. 2017 Feb. 28; 40(2): 90-99). Developmental assays include; growth rate, organ development (pharynx, vulva, cilia, etc.), fat storage, and dauer formation. Morphological assays include; length, width, area, volume, position, and optical density. Molecular assays (e.g., transcriptome assays, transcriptomic screens) can include; gene expression measured by QPCR of specific targets, gene expression measured by fluorescent reporter, gene expression measured by whole transcriptome sequencing (e.g., using RNAseq systems), protein expression, protein localization, and whole-genome sequencing. In these model organism functional assays, a ‘phenocopy’ of the human pathology of interest (animals exhibiting similar phenotypes to human) is not required, but rather pathogenicity in model organisms is discovered in the polyvariate space defined by features measured in these functional assays.

The choice of the phenotype and/or transcriptome features may be based on the understanding that each feature, when measured and normalized, contributed equally as an input variable for the classifier model. Thus, in certain embodiments, each phenotype or transcriptome feature in the panel is measured and normalized wherein none of the features are given any specific weight. In this instance each feature has a weight of 1.

In other embodiments, the choice of the phenotype and/or transcriptome features may be based on the understanding that each feature, when measured and normalized, contributed unequally as an input variable for the classifier model. In this instance, a particular feature in the panel can either be weighted as a fraction of 1 (for example if the relative contribution is low), a multiple of 1 (for example if the relative contribution is high) or as 1 (for example when the relative contribution is neutral compared to the other markers in the panel).

In still other embodiments, a machine learning system may analyze phenotype or transcriptome feature values without normalization of the values. Thus, the raw value obtained from the instrumentation to make the measurement may be analyzed directly.

Primary care healthcare practitioners, who may include physicians specializing in internal medicine or family practice as well as physician assistants and nurse practitioners, are among the users of the techniques disclosed herein, wherein they are the user that benefits from the output of the present classifier models. In embodiments, a clinical variant from a patient is identified and may be a variant of unknown significance or unassigned. As disclosed herein, the sequence of the clinical variant is used to generate the transgenic organism expressing the clinical variant. Those transgenic organisms are then subjected to one or more phenotype or transcriptome assays, which may yield one to hundreds (or more) of features that are measured and used as inputs for the classifier model. Those features are empirically determined and identified during the generation and training of the classifier model for a particular allele and associated specific human disease/condition.

The measured values of the phenotype and/or transcriptome features are used as input values for the first classifier model in a computer implemented system. An output value is obtained and compared to a pre-determined threshold value wherein the threshold is empirically determined during training of the classifier model and set to separate benign clinical variants from pathogenic clinical variants for a specific disease.

Once the physician or healthcare practitioner has a pathogenicity classification of the clinical variant for a specific disease, follow-up testing can be recommended for those with a pathogenic, or likely pathogenic, clinical variant. It should be appreciated that the precise cut off between likely benign and likely pathogenic, above which further testing is recommended may vary depending on many factors including, without limitation, (i) the desires of the patients and their overall health and family history, (ii) practice guidelines established by medical boards or recommended by scientific organizations, (iii) the physician's own practice preferences, and (iv) other information available for that particular allele and disease association.

In certain embodiments of the present invention, an apparatus is a computing device, for example, in the form of a computer or hand-held device that includes a processing unit, memory, and storage. The computing device can include or have access to a computing environment that comprises a variety of computer-readable media, such as volatile memory and non-volatile memory, removable storage and/or non-removable storage. Computer storage includes, for example, RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output, and/or a communication connection. The input can be one or several devices, such as a keyboard, mouse, touch screen, or stylus. The output can also be one or several devices, such as a video display, a printer, an audio output device, a touch stimulation output device, or a screen reading output device. If desired, the computing device can be configured to operate in a networked environment using a communication connection to connect to one or more remote computers. The communication connection can be, for example, a Local Area Network (LAN), a Wide Area Network (WAN) or other networks and can operate over the cloud, a wired network, wireless radio frequency network, and/or an infrared network.

Artificial intelligence systems include computer systems configured to perform tasks usually accomplished by humans, e.g., speech recognition, decision making, language translation, image processing and recognition, etc. In general, artificial intelligence systems have the capacity to learn, to maintain and access a large repository of information, to perform reasoning and analysis in order to make decisions, as well as the ability to self-correct.

Artificial intelligence systems may include knowledge representation systems and machine learning systems. Knowledge representation systems generally provide structure to capture and encode information used to support decision making. Machine learning systems are capable of analyzing data to identify new trends and patterns in the data. For example, machine learning systems may include neural networks, induction algorithms, genetic algorithms, etc. and may derive solutions by analyzing patterns in data.

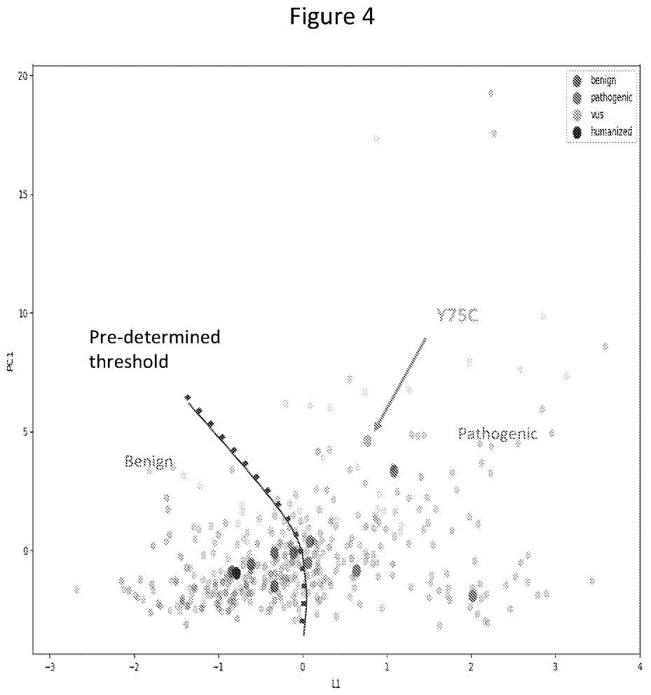

A variety of machine learning models are available, including support vector machines, decision trees, random forests, neural networks or deep learning neural networks. Generally, support vector machines (SVMs) are supervised learning models that analyze data for classification and regression analysis. SVMs may plot a collection of data points in n-dimensional space (e.g., where n is the number of phenotype and/or transcriptome features), and classification is performed by finding a hyperplane (decision boundary) that can separate the collection of data points into classes. See FIG. 4. In some embodiments, decision boundaries (e.g. pre-determined threshold) are linear, while in other embodiments, decision boundaries are non-linear. SVMs are effective in high dimensional spaces, are effective in cases in which the number of dimensions is higher than the number of data points, and generally work well on data sets with clear margins of separation.

Decision trees are a type of supervised learning algorithm also used in classification problems. Decision trees may be used to identify the most significant variable that provides the best homogenous sets of data. Decision trees split groups of data points into one or more subsets, and then may split each subset into one or more additional categories, and so forth until forming terminal nodes (e.g., nodes that do not split). Various algorithms may be used to decide where a split occurs, including a Gini Index (a type of binary split), Chi-Square, Information Gain, or Reduction in Variance. Decision trees have the capability to rapidly identify the most significant variables among a large number of variables, as well as identify relationships between two or more variables. Additionally, decision trees can handle both numerical and non-numerical data. This technique is generally considered to be a non-parametric approach, e.g., the data does not have to fit a normal distribution.

Random forest (or random decision forest) is a suitable approach for both classification and regression. In some embodiments, the random forest method constructs a collection of decision trees with controlled variance. Generally, for M input variables, a number of variables less than M is used to split groups of data points. The best split is selected, and the process is repeated until reaching a terminal node. Random forest is particularly suited to process a large number of input variables (e.g., thousands) to identify the most significant variables. Random forest is also effective for estimating missing data.

Neural nets (also referred to as artificial neural nets (ANNs)) are described herein. A neural net, which is a non-deterministic machine learning technique, utilizes one or more layers of hidden nodes to compute outputs. Inputs are selected and weights are assigned to each input. Training data is used to train the neural networks, and the inputs and weights are adjusted until reaching specified metrics, e.g., a suitable specificity and sensitivity.

ANNs may be used to classify data in cases in which correlation between dependent and independent variables is not linear or in which classification cannot be easily performed using an equation. More than 25 different types of ANNs exist, with each ANN yielding different results based on different training algorithms, activation/transfer functions, number of hidden layers, etc. In some embodiments, more than 15 types of transfer functions are available for use with the neural network. Prediction of the clinical variant pathogenicity is based upon one or more of the type of ANN, the activation/transfer function, the number of hidden layers, the number of neurons/nodes, and other customizable parameters.

Deep learning neural networks, another machine learning technique, are similar to regular neural nets, but are more complex (e.g., typically have multiple hidden layers) and are capable of automatically performing operations (e.g., feature extraction) in an automated manner, generally requiring less interaction with a user than a traditional neural net.

In some embodiments, inputs may be selected in order to improve the performance of the classifier model. For example, rather than picking the set of inputs that achieves the highest possible sensitivity with a pathogenicity relevant specificity such as 80% or greater, the inputs are selected to reach a sensitivity threshold (e.g., 80% or greater), and once reaching this threshold, the inputs are selected to optimize performance of the classifier model, thereby improving the performance of the classifier model.

Accordingly, systems, methods and computer readable media are presented herein regarding using a machine learning system, e.g. to generate a classifier model, to classify a clinical variant (e.g. from a patient) into a pathogenicity class of pathogenic or benign. A set of data comprising a plurality of individual records, each individual record including a plurality of phenotype and/or transcriptome features corresponding to a clinical variant, and wherein the set of data also includes a diagnostic indicator indicating whether or not the clinical variant is pathogenic or benign based on originating patient data is stored in a memory, accessible by the classifier model or machine learning system. The plurality of parameters includes phenotype and/or transcriptome features and other factors which may be selected as inputs into the classifier model. The diagnostic indicator is an affirmative indicator that the patient with a particular clinical variant (e.g. pathogenic) was diagnosed with the associated specific disease/condition. A subset of the plurality of parameters is selected for inputs into the machine learning system, wherein the subset includes a panel of at least four different measure phenotype and/or transcriptome features.

In some embodiments, although the machine learning system can evolve over time to make more accurate predictions, the machine learning system may have the capability to deploy improved predictions on a scheduled basis. In other words, the techniques used by the machine learning system to determine pathogenicity classification may remain static for a period of time, allowing consistency with regard to determination of classification. At a specified time, the machine learning system may deploy updated techniques that incorporate analysis of new data to produce an improved classification. Thus, the machine learning systems described herein may operate: (1) in a static manner; (2) in a semi-static manner, in which the classifier is updated according to a prescribed schedule (e.g., at a specific time); or (3) in a continuous manner, being updated as new data is available.

In some embodiments, this disclosure provides computer-implemented methods comprising: a) obtaining, by one or more processors, a data set comprising measured phenotype features and/or transcriptome features of a transgenic organism expressing a human clinical variant, wherein the phenotype features are from a population of clinical variants, wherein the clinical variants are labeled with a diagnostic indicator of pathogenic or benign for a specific human disease; b) selecting a subset of the measured phenotype and/or transcriptome features for inputs into a machine learning system, wherein the subset includes transcriptome features and/or at least four phenotype features, and/or and the diagnostic indicator for the clinical variants; c) randomly partitioning the data set in training data and validation data; and, d) generating a classifier model using a machine learning system based on the training data and the subset of inputs, wherein each input has an associated weight, and wherein the classifier model provides binary outcomes selected from pathogenic or likely pathogenic above a pre-determined threshold or benign or likely benign below a pre-determined threshold. In some embodiments, the classifier model is generated using a machine learning system based on the training data and the subset of inputs, each of which include measured phenotype features and measured transcriptome features. In some embodiments, the method further comprises: (1) obtaining one or more test results from the diagnostic testing which confirm or deny the presence of the human disease; (2) incorporating the one or more test results into the first training data for further training of the first classifier model of the machine learning system; and, (3) generating an improved first classifier model by the machine learning system.

In some embodiments, this disclosure provides methods, in a computer implemented system comprising at least one processor and at least one memory, the at least one memory comprising instructions executed by the at least one processor to cause the at least one processor to implement one or more classifier models to predict pathogenicity for a clinical variant of a human disease, comprising: a) obtaining measured phenotype features and/or transcriptome features of a transgenic organism expressing the human clinical variant; b) classifying the clinical variant into a pathogenicity category of pathogenic or likely pathogenic using a first classifier model, wherein the first classifier model is generated by a machine learning system using a first training data set that comprises phenotype features and/or transcriptome features from the transgenic organism of a panel of transcriptome features and/or at least four phenotype features from a population of clinical variants, wherein the clinical variants are labeled with a diagnostic indicator of pathogenic or benign for the human disease; wherein the first classifier model classifies the clinical variant in a pathogenicity category of pathogenic or likely pathogenic using input variables of the measured phenotype features of a panel of phenotype features, and/or a panel of measure transcriptome features from a panel of transcriptome features, from the transgenic organism when an output of the first classifier model is above a predetermined threshold; and, c) providing notification to a user for patient testing when the clinical variant is predicted to be pathogenic or likely pathogenic for a human disease. In some embodiments, the first classifier model classifies the clinical variant in a pathogenicity category of pathogenic or likely pathogenic using input variables of measured phenotype features and measured transcriptome features.

In some embodiments, the predetermined threshold can be determined by linear discriminator analysis (LDA) comprising establishing a classifier algorithm by overlaying known pathogenic and benign variants as training constraints on an LDA algorithm, and overlaying variants of unknown significance (VUS) on an LDA plot generated by the LDA. In some embodiments, the methods can comprise generating a receiver operator curve (ROC) of the magnitude of values for the known pathogenic and benign variants and determining an optimal cutoff providing a sensitivity and specificity of at least about 70%, wherein optionally at least one of the sensitivity or specificity is at least about 80%, preferably at least about 90%, and more preferably at least about 95%. In some embodiments, the threshold is determined based on the performance of the distance values for each variant in the LDA from the wt allele, such as a cut of value, or threshold, wherein the classifier model has a performance of at least 0.70 specificity as demonstrated in a ROC. See FIG. 9.

In some embodiments, this disclosure provides methods for identifying one or more human clinical variants as pathogenic, likely pathogenic, benign, or likely benign by measuring at least four measured phenotype features of a non-human transgenic organism expressing at least one of the human clinical variants (e.g., a gene-swapped humanized C. elegans animal as illustrated in FIG. 9a (the corresponding gene of the C. elegans genome could also be directly modified to match the sequence of the human clinical variant). In such embodiments, the data can be organized as a Linear Discriminator Plot (LDP) as illustrated in FIG. 9b. Each point in the LDP represents an aggregate of the phenotype features (e.g., an aggregate of 26 features) that were measured for each clinical variant, and each is measured relative to the “wild-type” (wt) version of the human clinical variant which in certain embodiments is a chimera of all the most common alleles at any given amino acid position of the heterogenous human clinical variants of a particular gene. The wt version is the zero position from which all other variants can be measured in distance from that point. Those measured distances form the basis for determining a “threshold” via performance that can be measured in a Receiver Operator Curve (ROC) (e.g., as illustrated in FIG. 9c). From this data, the performance (specificity and sensitivity) of that human clinical variant (e.g., variant of unknown significance (“VUS”)) can be determined (e.g., as illustrated in FIG. 9d). Thus, in such embodiments, the distance of each tested value (i.e., the measured phenotype features) from wt in the LDP can be used to train the classifier model.

In some embodiments, the diagnostic indicator is selected from pathogenic, likely pathogenic, likely benign and benign. In some embodiments, the human disease is selected from epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, or Tay-Sachs disease. In some embodiments, the human disease is selected from neuromuscular, epilepsy, ataxia, dystonia, neurodegeneration, cancer, or metabolic disease or condition. In some embodiments, the clinical variant is a variant of unknown or uncertain significance or unassigned. In some embodiments, the transgenic organism is a nematode or zebrafish. In some embodiments, the phenotype features are measured in an electropharyngeogram (EPG) assay, morphology and/or movement phenotype assay, or a gene expression profile, lethality, incidence of males, axonal outgrowth, or synaptic transmission assay. In some embodiments, the phenotype features are selected from pharyngeal pumping duration, inter-pump interval, pumping frequency, peak amplitude of different pump components, speed, forward vs. reverse travel, curling, length, or width. In some embodiments, the first training data comprises values from a panel of at least five phenotype features. In some embodiments, the first training data further comprises patient phenotype, patient drug response, or phenotype in a second transgenic organism expressing the human clinical variant, wherein the second transgenic organism is selected from frog oocyte, fly, rodent, or induced pluripotent stem cell (iPSC)-derived cells. In some embodiments, the input variables comprise measured phenotype features from a panel of at least five phenotype features. In some embodiments, the machine learning system further comprises iteratively regenerating the first classifier model by training the first classifier model with new training data to improve the performance of the first classifier model. In some embodiments, the first classifier model comprises a support vector machine, a decision tree, a random forest, a neural network, a deep learning neural network, pattern recognition, or a logistic regression algorithm. In some embodiments, observation of a measured phenotype in at least about 75% of transgenic organisms indicates the clinical variant is pathogenic, optionally wherein the measured phenotype is lethality, and optionally wherein the presence of the clinical variant in the transgenic organism(s) is confirmed by nucleotide sequencing. In some embodiments, the transgenic organism expresses the human clinical variant following modification to create the human clinical variant in the genome of the transgenic organism using CRISPR, and optionally wherein the presence of the clinical variant in the transgenic organism(s) is confirmed by nucleotide sequencing. Other embodiments are also contemplated herein as would be understood by those of ordinary skill in the art.

In some embodiments, this disclosure provides a computer-implemented method comprising: a) obtaining, by one or more processors, a data set comprising measured transcriptome features of a transgenic organism expressing a human clinical variant, wherein the transcriptome features are from a population of clinical variants, wherein the clinical variants are labeled with a diagnostic indicator of pathogenic or benign for a specific human disease; b) selecting a subset of the measured transcriptome features for inputs into a machine learning system, wherein the subset includes transcriptome features and the diagnostic indicator for the clinical variants; c) randomly partitioning the data set in training data and validation data; and, d) generating a classifier model using a machine learning system based on the training data and the subset of inputs, wherein each input has an associated weight, and wherein the classifier model provides binary outcomes selected from pathogenic or likely pathogenic above a pre-determined threshold or benign or likely benign below a pre-determined threshold. In some embodiments, this disclosure provides a method, in a computer implemented system comprising at least one processor and at least one memory, the at least one memory comprising instructions executed by the at least one processor to cause the at least one processor to implement one or more classifier models to predict pathogenicity for a clinical variant of a human disease, comprising: a) obtaining measured transcriptome features of a transgenic organism expressing the human clinical variant; b) classifying the clinical variant into a pathogenicity category of pathogenic or likely pathogenic using a first classifier model, wherein the first classifier model is generated by a machine learning system using a first training data set that comprises transcriptome features from the transgenic organism of a panel of transcriptome features from a population of clinical variants, wherein the clinical variants are labeled with a diagnostic indicator of pathogenic or benign for the human disease; wherein the first classifier model classifies the clinical variant in a pathogenicity category of pathogenic or likely pathogenic using input variables of the measured transcriptome features of a panel of transcriptome features from the transgenic organism when an output of the first classifier model is above a predetermined threshold; and, c) providing notification to a user for patient testing when the clinical variant is predicted to be pathogenic or likely pathogenic for a human disease. In some embodiments, the diagnostic indicator is selected from pathogenic, likely pathogenic, likely benign and benign. In some embodiments, the human disease is selected from epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, or Tay-Sachs disease. In some embodiments, the human disease is selected from neuromuscular, epilepsy, ataxia, dystonia, neurodegeneration, cancer, or metabolic disease or condition. In some embodiments, the clinical variant is a variant of unknown or uncertain significance or unassigned. In some embodiments, the transgenic organism is a nematode or zebrafish. In some embodiments, the first training data comprises values from a panel of transcriptome features. In some embodiments, the first training data further comprises patient transcriptome features, patient drug response, or transcriptome features in a second transgenic organism expressing the human clinical variant, wherein the second transgenic organism is selected from frog oocyte, fly or rodent. In some embodiments, the input variables comprise measured transcriptome features from a panel transcriptome features. In some embodiments, the machine learning system further comprises iteratively regenerating the first classifier model by training the first classifier model with new training data to improve the performance of the first classifier model. In some embodiments, the first classifier model comprises a support vector machine, a decision tree, a random forest, a neural network, a deep learning neural network, pattern recognition, or a logistic regression algorithm. In some embodiments, the method further comprises: (1) obtaining one or more test results from the diagnostic testing which confirm or deny the presence of the human disease; (2) incorporating the one or more test results into the first training data for further training of the first classifier model of the machine learning system; and, (3) generating an improved first classifier model by the machine learning system. In some embodiments, the classifier model is generated using a machine learning system based on the training data and the subset of inputs, each of which include measured phenotype features and measured transcriptome features. In some embodiments, the first classifier model classifies the clinical variant in a pathogenicity category of pathogenic or likely pathogenic using input variables of measured phenotype features and measured transcriptome features.

Thus, in some embodiments, this disclosure provides a computer-implemented method comprising: a) obtaining, by one or more processors, a data set comprising measured phenotype features of a transgenic organism expressing a human clinical variant, wherein the phenotype features are from a population of clinical variants, wherein the clinical variants are labeled with a diagnostic indicator of pathogenic or benign for a specific human disease; b) selecting a subset of the measured phenotype features for inputs into a machine learning system, wherein the subset includes at least four phenotype features and the diagnostic indicator for the clinical variants; c) randomly partitioning the data set in training data and validation data; and, d) generating a classifier model using a machine learning system based on the training data and the subset of inputs, wherein each input has an associated weight, and wherein the classifier model provides binary outcomes selected from pathogenic or likely pathogenic above a threshold value or benign or likely benign below a threshold value; optionally wherein either or both of the threshold values are pre-determined. In some embodiments, this disclosure provides a method, in a computer implemented system comprising at least one processor and at least one memory, the at least one memory comprising instructions executed by the at least one processor to cause the at least one processor to implement one or more classifier models to predict pathogenicity for a clinical variant of a human disease, comprising: a) obtaining measured phenotype features of a transgenic organism expressing the human clinical variant; b) classifying the clinical variant into a pathogenicity category of pathogenic or likely pathogenic using a first classifier model, wherein the first classifier model is generated by a machine learning system using a first training data set that comprises phenotype features from the transgenic organism of a panel of at least four phenotype features from a population of clinical variants, wherein the clinical variants are labeled with a diagnostic indicator of pathogenic or benign for the human disease, wherein the first classifier model classifies the clinical variant in a pathogenicity category of pathogenic or likely pathogenic using input variables of the measured phenotype features of a panel of phenotype features from the transgenic organism when an output of the first classifier model is above a threshold value, optionally wherein threshold value is predetermined; and, c) optionally providing notification to a user for patient testing when the clinical variant is predicted to be pathogenic or likely pathogenic for a human disease. In some embodiments, this disclosure provides a computer-implemented method comprising: a) obtaining, by one or more processors, a data set comprising measured phenotype features of a transgenic organism expressing a human clinical variant, wherein the phenotype features are from a population of human clinical variants, wherein the human clinical variants are labeled with a diagnostic indicator of pathogenic or benign for a specific human disease; b) selecting a subset of the measured phenotype features for inputs into a machine learning system, wherein the subset includes at least four phenotype features and the diagnostic indicator for the human clinical variants; c) randomly partitioning the data set into training data and validation data; d) generating a classifier model using a machine learning system based on the training data and the subset of inputs, wherein each input has an associated weight, and wherein the classifier model provides binary outcomes selected from pathogenic or likely pathogenic above a threshold or benign or likely benign below the threshold; and, e) obtaining at least four measured phenotype features of a transgenic organism expressing at least one of the human clinical variants and classifying the clinical variant as pathogenic, likely pathogenic, benign, or likely benign based on the measured phenotype features of the transgenic organism; and, f) optionally providing notification to a patient expressing the clinical variant(s) when the clinical variant is predicted to be pathogenic or likely pathogenic for a human disease. In some such embodiments, the diagnostic indicator can be selected from pathogenic, likely pathogenic, likely benign and benign. In some embodiments, the human disease can be selected from epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemia, xeroderma pigmentosum, Fanconi's anemia, retinitis pigmentosa, ataxia telangiectasia, Bloom's syndrome, retinoblastoma, or Tay-Sachs disease. In some embodiments, the human disease is selected from the group consisting of neuromuscular, epilepsy, ataxia, dystonia, neurodegeneration, cancer, and a metabolic disease or condition. In some embodiments, the at least one clinical variant can be a variant of unknown or uncertain significance or unassigned. In some embodiments, the transgenic organism can be a nematode or zebrafish. In some embodiments, the phenotype features can be measured in an electropharyngeogram (EPG) assay, morphology and/or movement phenotype assay, or a gene expression profile, lethality, incidence of males, axonal outgrowth, or synaptic transmission assay. In some embodiments, the phenotype features can be selected from pharyngeal pumping duration, inter-pump interval, pumping frequency, peak amplitude of different pump components, speed, forward vs. reverse travel, curling, length, width, lethality, attenuation, bending angle-mid-point asymmetry, maximum amplitude (um), self-contact distance, mean amplitude (um), body wave number, area, dynamic amplitude (stretch), center point speed (um/s), center point trajectory/time, peristaltic speed (um/s), absolute peristaltic track length/time, activity, brush stroke, length, reverse swim, curling, fit, swimming speed, wave initiation rate, wavelength, width, proportion time forward, proportion time reverse, straight-line speed, forward speed, and reverse speed. In some embodiments, the first training data can comprise values from a panel of at least five phenotype features. In some embodiments, the first training data further comprises patient phenotype, or phenotype in a second transgenic organism expressing the human clinical variant, wherein the second transgenic organism is selected from frog oocyte, nematode or zebrafish, fly or rodent or iPSC cells. In some embodiments, the transgenic organism and the second transgenic organism are different, optionally wherein the transgenic organism is a nematode and the second transgenic organism is zebrafish. In some embodiments, the input variables comprise measured phenotype features from a panel of at least four, or at least five phenotype features, optionally about six to about eight phenotype features, about nine to about 15 phenotype features, or about 16 to about 30 phenotype features. In some embodiments, the machine learning system can further comprise iteratively regenerating the first classifier model by training the first classifier model with new training data to improve the performance of the first classifier model. In some embodiments, the first classifier model can comprise a support vector machine, a decision tree, a random forest, a neural network, a deep learning neural network, pattern recognition, or a logistic regression algorithm. In some embodiments, the method(s) can comprise: (1) obtaining one or more test results from the diagnostic testing which confirm or deny the presence of the human disease; (2) incorporating the one or more test results into the first training data for further training of the first classifier model of the machine learning system; and, (3) generating an improved first classifier model by the machine learning system. In some embodiments, the transgenic organism expresses the human clinical variant following modification to create the human clinical variant in the genome of the transgenic organism, optionally using CRISPR, and/or replacing the naturally-occurring coding sequence of the transgenic organism with a modified coding sequence; optionally wherein the presence of the clinical variant in the transgenic organism(s) is confirmed by nucleotide sequencing.

In some embodiments, this disclosure provides a computer-implemented method comprising: a) obtaining, by one or more processors, a data set comprising measured transcriptome features of a transgenic organism expressing at least one human clinical variant, wherein the transcriptome features are from a population of clinical variants, wherein the clinical variants are labeled with a diagnostic indicator of pathogenic or benign for a specific human disease; b) selecting a subset of the measured transcriptome features for inputs into a machine learning system, wherein the subset includes transcriptome features and the diagnostic indicator for the clinical variants; c) randomly partitioning the data set in training data and validation data; and, d) generating a classifier model using a machine learning system based on the training data and the subset of inputs, wherein each input has an associated weight, and wherein the classifier model provides binary outcomes selected from pathogenic or likely pathogenic above a pre-determined threshold or benign or likely benign below a pre-determined threshold. In some embodiments, this disclosure provides a method, in a computer implemented system comprising at least one processor and at least one memory, the at least one memory comprising instructions executed by the at least one processor to cause the at least one processor to implement one or more classifier models to predict pathogenicity for a clinical variant of a human disease, comprising: a) obtaining measured transcriptome features of a transgenic organism expressing the human clinical variant; b) classifying the clinical variant into a pathogenicity category of pathogenic or likely pathogenic using a first classifier model, wherein the first classifier model is generated by a machine learning system using a first training data set that comprises transcriptome features from the transgenic organism of a panel of transcriptome features from a population of clinical variants, wherein the clinical variants are labeled with a diagnostic indicator of pathogenic or benign for the human disease, wherein the first classifier model classifies the clinical variant in a pathogenicity category of pathogenic or likely pathogenic using input variables of the measured transcriptome features of a panel of transcriptome features from the transgenic organism when an output of the first classifier model is above a predetermined threshold; and, c) providing notification to a user for patient testing when the clinical variant is predicted to be pathogenic or likely pathogenic for a human disease. In some embodiments, the diagnostic indicator can be selected from the group consisting of pathogenic, likely pathogenic, likely benign and benign. In some embodiments, the human disease can be selected from the group consisting of epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, or Tay-Sachs disease. In some embodiments, the human disease can be selected from the group consisting of neuromuscular, epilepsy, ataxia, dystonia, neurodegeneration, cancer, and a metabolic disease or condition. In some embodiments, the clinical variant can be a variant of unknown or uncertain significance (VUS) or unassigned. In some embodiments, the transgenic organism can be a nematode or zebrafish. In some embodiments, the first training data can comprise values from a panel of transcriptome features. In some embodiments, the first training data further can comprise patient transcriptome features, patient drug response, or transcriptome features in a second transgenic organism expressing the human clinical variant, wherein the second transgenic organism can be selected from frog oocyte, fly or rodent. In some embodiments, the input variables can comprise measured transcriptome features from a panel transcriptome features. In some embodiments, the machine learning system can further comprise iteratively regenerating the first classifier model by training the first classifier model with new training data to improve the performance of the first classifier model. In some embodiments, the first classifier model can comprise a support vector machine, a decision tree, a random forest, a neural network, a deep learning neural network, pattern recognition, or a logistic regression algorithm. In some embodiments, the method(s) can further comprise: (1) obtaining one or more test results from the diagnostic testing which confirm or deny the presence of the human disease; (2) incorporating the one or more test results into the first training data for further training of the first classifier model of the machine learning system; and, (3) generating an improved first classifier model by the machine learning system.

In some embodiments of the methods disclosed herein, the classifier model can be generated using a machine learning system based on the training data and the subset of inputs, each of which include measured phenotype features and measured transcriptome features. In some embodiments, the first classifier model can classify the clinical variant in a pathogenicity category of pathogenic or likely pathogenic using input variables of measured phenotype features and measured transcriptome features. In some embodiments, the classifier can use one threshold value for discriminating pathogenic or likely pathogenic from benign or likely benign. In some embodiments, a range of threshold values is selected from the group consisting of direct outputs from either a radial or linear classifier, cartesian coordinates from an origin on dimension reduced plots, and composite euclidean vector magnitudes from multidimensional feature sets. In some embodiments, the threshold value is determined by Receiver Operator Curve (ROC) curve analysis. In some embodiments, the classifier can create two threshold values using Firth logistic regression. In some embodiments, the threshold values can comprise a lower threshold and a upper threshold, wherein the lower threshold is the maximum threshold for a clinical variant being identified as benign and the upper threshold is the minimal threshold for being identified as pathogenic.

Other embodiments are also contemplated herein as would be understood by those of ordinary skill in the art.

Certain embodiments are further described in the following examples. These embodiments are provided as examples only and are not intended to limit the scope of the claims in any way.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Development of a Multi-Feature Model Based on Electrophysiological Phenotype for Classifying Clinical Variants of Unknown Significance as to Pathogenicity

Provided herein is multi-feature classification model and method for identifying clinical variants of unknown significance as pathogenic or likely pathogenic based on features derived from an electrophysiological assay.

The phenotypic datasets from functional assays are integrated into a feature-extraction and variant classification pipeline. The present classifier model was generated using clinical variants of Syntaxin binding protein 1 (STXBP1), which is a protein involved in synaptic vesicle trafficking. Autosomal dominant mutations in STXBP1 are implicated in childhood epilepsies and several neurodevelopmental disorders. Humanized nematodes were prepared according to methodologies disclosed in U.S. Ser. No. 16/281,988, herein incorporated by reference. Briefly, the STXBP1 wild type gene was installed into the native unc-18 locus using CRISPR-mediated gene-swap methods. Next clinical variants (pathogenic and benign) were installed into the wild type gene via site directed mutagenesis.

### Dataset

For the STXBP1, our dataset was composed of electropharyngeogram EPG data from transgenic nematodes harboring the human STXBP1 coding sequence installed as gene substitutions at the orthologous gene position of the C. elegans. Based on the sequence installed, the transgenic nematodes were separated into a benign group which consists of known benign variant V841 and the human canonical sequence, and a pathogenic group which consists of known pathogenic variants R122X and R406H.

### Feature Extraction

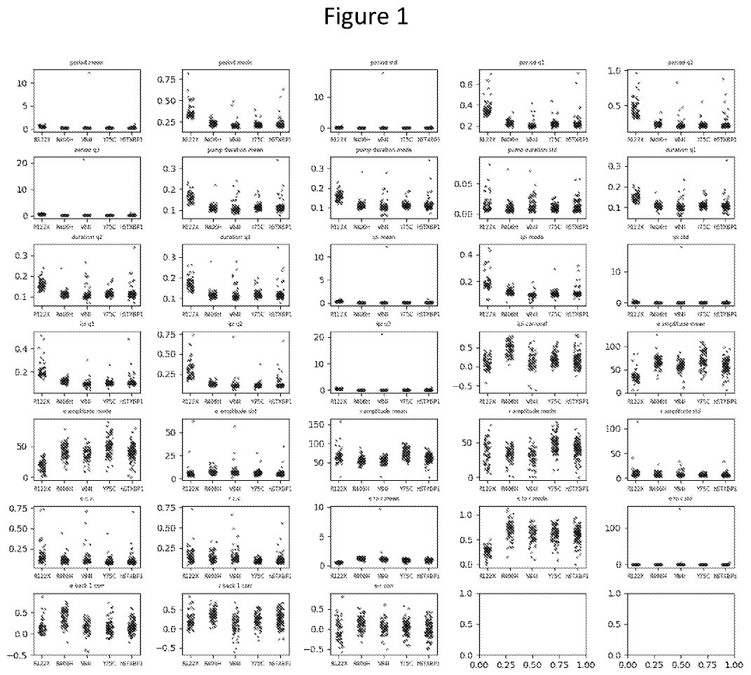

In the electropharyngeogram (EPG) assay, time-varying membrane current was recorded at a sampling rate of 500 Hz. Signal was band-stop filtered to remove electrical noise. Additional high-pass or low-pass filters were applied as needed to enhance signal quality and the prominence of pharyngeal pump subcomponents for detection. Peak-detection algorithm was applied and for each pump waveform, the largest positive and negative spike was detected and labeled as the ‘E’ and ‘R’ component, respectively. Based on the timing and the amplitude of these pump waveform components, we quantified and extracted 33 separate numerical parameters (e.g. phenotype features) as a measurement of the pharyngeal pumping behavior from each worm. See FIG. 1. To remove noise and collinearity from our multivariate parameter space, we employed dimension-reduction (such as principal component analysis) (PCA)) to distill the data down to a few most salient features, which were linear combinations of all the available parameters.

### Classification

In order to apply what we learn from known pathogenic vs. benign variants to variants of unknown significance (VUS), we used these extracted features from our dataset of labeled pathogenic and benign worms to train supervised learning classifiers. We performed cross-validation and evaluated the performance of the classifier on hold-out data, here we report the performance scores as weighted averages for both benign and pathogenic groups. Linear discriminant analysis (LDA), a linear classification algorithm, achieved a recall score of 0.849, a precision score of 0.849, and an f1 score of 0.848. Support vector machine with linear kernel (Linear SVM, another linear classification algorithm), achieved a recall score of 0.852, a precision score of 0.853, and an f1 score of 0.852. Support vector machine with radial basis function kernel, a nonlinear classification algorithm, achieved a recall score of 0.830, a precision score of 0.843, and an f1 score of 0.830.

This classification accuracy is high enough at a single worm level to confidently tell apart different strains of worms with different variants for this gene, if the unknown variant falls into the mapped pathogenic vs. benign space. With more known pathogenic and benign variants installed and assayed, we would map out this functional feature space more thoroughly and the decision boundary (e.g. pre-determined threshold) between benign and pathogenic subspace defined. So that when a new known variant is assessed, a classification of pathogenicity can be derived based on where in the feature space this new variant is assigned.

### Example 2: Development of a Multi-Feature Model Based on Mobility and Morphology Phenotypes for Classifying Clinical Variants of Unknown Significance as to Pathogenicity

Provided herein is multi-feature classification model and method for identifying clinical variants of unknown significance as pathogenic or likely pathogenic based on phenotypes derived from a movement and morphology assay.

The phenotypic datasets from functional assays are integrated into a feature-extraction and variant classification pipeline. The present classifier model was generated using clinical variant of Syntaxin binding protein 1 (STXBP1), which is a protein involved in synaptic vesicle trafficking. Autosomal dominant mutations in STXBP1 are implicated in childhood epilepsies and several neurodevelopmental disorders. Humanized nematodes were prepared according to methodologies disclosed in US Patent Publ. No. 2020/0060246, herein incorporated by reference. Briefly, the STXBP1 wild type gene was installed into the native unc-18 locus using CRISPR-mediated gene-swap methods. Next clinical variants (pathogenic and benign) were installed into the wild type gene via site directed mutagenesis.

### Dataset

For the STXBP1, our dataset composed of movement assay data from worms harboring the transgenic nematode STXBP1 coding sequence installed as gene substitutions at the orthologous gene position of the C. elegans. Based on the sequence installed, transgenic nematodes were categorized into a benign group which consists of known benign variants and the human canonical sequence, and a pathogenic group which consists of known pathogenic variants. See FIG. 2.

### Feature Extraction

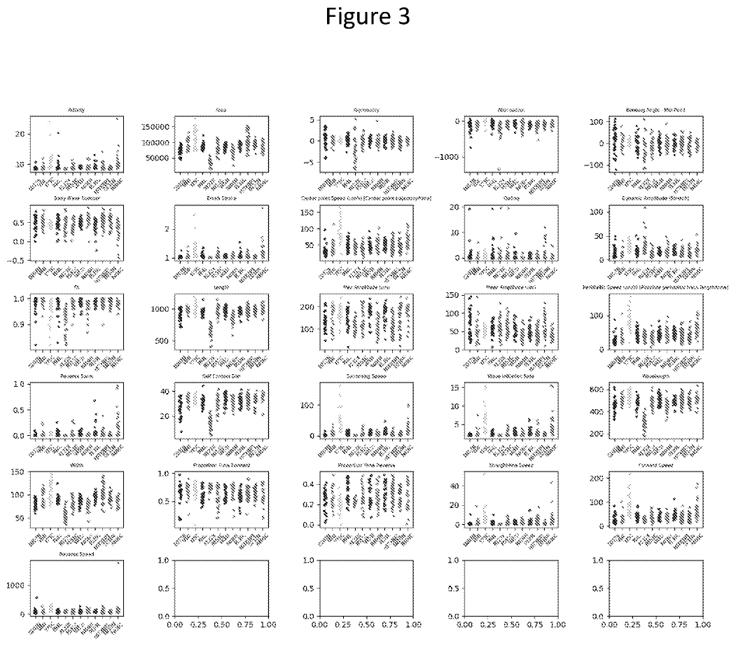

In the movement assay, high resolution videos were acquired at a frame rate of 14 Hz. We used commercial software to quantify and extract 26 separate numerical parameters (e.g. phenotype features) measuring different aspects of the movement and morphology from each transgenic nematode. See FIG. 3. To remove noise and collinearity from our multivariate parameter space, we employed dimension-reduction (such as PCA) to distill the data down to a few most salient features, which were linear combinations of all the available parameters. Additionally, linear discrimination was performed to take into account the class information.

### Classification

Linear discrimination analysis (LDA) was performed on the PCA-transformed phenotypic features from known pathogenic and benign variants. To illustrate that this type of simple linear classification was able to separate the known pathogenic variants from the known benign variants, we plotted the first principal component from PCA against the first dimension of LDA using all the data. See FIG. 4.

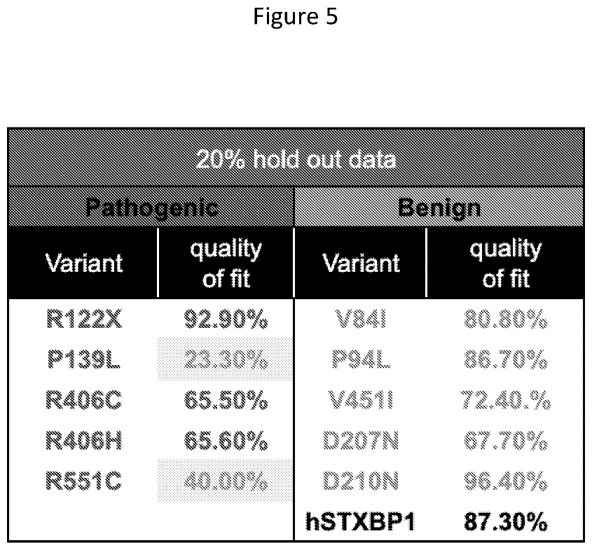

We performed cross-validation and evaluated the performance of the classifier on hold-out data, here we report the classification accuracy for each labeled strain as whether individual worms from this strain were correctly predicted to be benign or pathogenic by an LDA classifier. See FIG. 5. This analysis was used to classify a VUS as pathogenic. See FIG. 4.

### Example 3: F0 Crispinlet Knock-In System for Classifying Clinical Variants of Uncertain or Unknown Significance as to Pathogenicity

In some embodiments, zebrafish can be modified using the “F0 Crispinlet knock-in” system described herein to express a clinical variant gene and assayed to determine, or estimate, the pathogenicity of that clinical variant gene by a phenotypic assay. This method is a CRISPR-based lethality assay in which transgenic zebrafish embryos are modified to express a clinical variant gene and assayed for a phenotypic change (e.g., lethality). In some embodiments, a threshold level of phenotypic change in a population of such transgenic zebrafish indicates the clinical variant is pathogenic. For instance, a pathogenic clinical variant may be one that results in 75% or more lethality (i.e., 25% or less survival) in a population of such transgenic zebrafish. Such methods provide simple, fast and affordable functional testing for detecting pathogenicity in patient-observed clinical variants (i.e., gene variants). In some embodiments, such clinical variants are Variants of Uncertain Significance (VUS), which are a recognized problem that interfere with diagnostic rates and discovery of personalized therapeutics. When studying clinical sequences on populations, the rate of diagnosis of a genetic cause is dependent on ratio of pathology to VUS. As a VUS becomes determined to be pathologic (e.g., the expectation is about ⅓), the increase in pathological assessment enables higher “diagnostic rate” in the patient populations. Functional testing, such as the phenotypic assays described herein can be used to determine whether a VUS is either pathogenic or benign. The pathogenicity testing in zebrafish embryos described in this example provides data that serves as a “training data set” for the computerized (e.g., machine learning) systems described herein.

The aforementioned SCN1A gene has an established variant pathogenicity in humans (PMID: 20301494 (Miller, et al. SCNA1 Seizure Disorders, GeneReviews, 2019)). While there are 1328 missense variants that have been observed in the SCN1A coding sequence (www.ncbi.nlm nih.gov), only 25% of the observed variants have been assigned pathogenicity status (175× P; 159× LP) and only 0.6% annotated as benign (1× B; 7× LB). The remaining 74% of variants are either conflicted (47×), assigned as VUS (320×), or remain not annotated (619×). SCN1A variants exhibit a monogenic propensity for epilepsy in an autosomal dominant manner (https://omim.org/entry/182389). Examination of a large sampling of OMIM entries indicates autosomal-recessive (AR) diseases are only 2× more frequent that autosomal-dominant (AD) disease (REF clinphen blog). Of the 4346 genes in OMIM with disease association, half are associated with AR, one quarter are AD, and the remainder are mixed, unassigned, or X-linked. The result is close to 1000 disease-associated genes will have variants that manifest with autosomal dominant behavior. Some of these variants will be gain-of-function alleles, but a large portion can be expected to cause pathogenicity via haploinsufficiency mechanism. For instance, 300 genes have been reported in the literature to have haploinsufficiency as the cause of disease (PMID: 18523451) and the actual number may be 3× higher (PMID: 20976243). On essentiality, recent estimates are converging on 10% of the 20,000 protein-coding genes as essential (PMID: 315041710). Many of these essential genes are sensitive to dosage and will be in the gene group that exhibits haploinsufficiency as the cause of disease. Knockdown of expression of SCN1A in zebrafish leads to seizure behavior due to haploinsufficiency (PMID: 29915537 (Griffin, et al. Front Pharmacol. 2018 Jun. 4; 9:573). Many of these essential genes are sensitive to dosage and will be in the gene group that exhibits haploinsufficiency as the cause of disease. Multiple epilepsy targets in addition to SCN1A, such as SCN2A, SCN8A, KCNQ2, GRIN2A, SLC2A1, and STXBP1, are also known, and could be studied using the F0 crispinlet knock-in assay system.

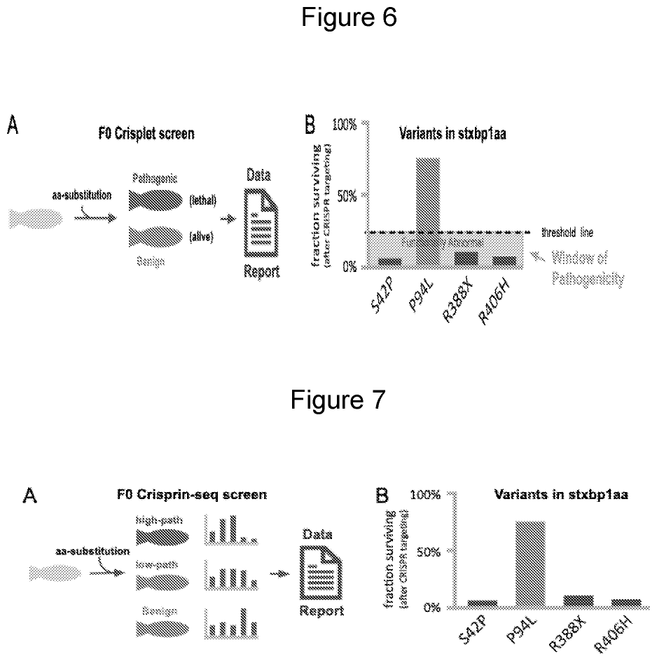

The F0 crispinlet knock-in assay described herein, and referred to as a CRISPR/Cas9-editing F0 lethality assay can be used to detect pathogenicity in a zebrafish vertebrate model. It is known that embryos injected with CRISPR editing reagents for targeted allele conversion show a range of tissue conversion efficiencies. For instance, some cells are bi-allelically converted, some are haplotype converted, while others are not converted at all. The levels of mosaicism in an embryo injection will be impacted by cell division stage and the happenstance of injection quality. Additionally, the efficiency of the chosen guide RNA and the composition of the donor homology oligonucleotide (dhODN) donor homology template will impact levels of somatic conversion. The result for a set of embryos injected with CRISPR/Cas9 editing reagents (“F0 crispinlet knock-ins”) is a range of conversion efficiencies per embryo. For an essential gene, a high degree of biallelic conversion to a non-functional variant will frequently result in death of the embryo. For genes where biallelic loss-of-function results in non-viability, the levels of embryo lethality provide a correlative measure of variant pathogenicity (FIG. 6A). Injection mixtures targeting creation of a non-functional “lethality-as-homozygote” variant(s) result in large numbers of embryo deaths, while insertion of a benign variant(s) does not. For instance, injection of STXBP1 variants S42P, R388X, and R406H result in a high rate of embryo death, while the lethality of injection of the benign SCN1A variant P94L is much lower (FIG. 6B). The level of embryo death in a set of embryo injections becomes an indicator of variant pathogenicity. In some embodiments, there is a threshold of lethality under which there will be a “window” of non-viability for a variant that induces lethality-as-homozygote pathogenicity.

In one embodiment, genetic variants in SCN1A are used in the F0 crispinlet knock-in assay system to determine their propensity for pathogenicity. To achieve this goal, data is analyzed from zebrafish embryo injected CRISPR/Cas9 editing reagents and the “window of pathogenicity” is revealed for which pathogenic variants in SCN1A cause high levels of embryo lethality. In embodiments, the window of pathogenicity, which is also referred to herein as a threshold is determined based on the input of the analyzed data (e.g. did the embryos live or die) wherein for each injection the percent of embryos that died are recorded. In certain embodiments, the threshold value may be set at 75% lethality. In other words, if 75%, or more, of injected embryos with a specific clinical variant die the clinical variant is classified as pathogenic or likely pathogenic. The resulting embryos can be sequenced, or other functional assays performed for embryos that survived to confirm pathogenicity. Use of a Receiver Operator Curve (ROC) can be used to determine the sensitivity and specificity of the threshold value which can then inform if the threshold value should be moved to increase the percentage of true pathogenic classification of clinical variants. As understood by those skilled in the art, ROC curves can be used to determine the performance of a test, but that input factors from the data set are used to generate a classifier model which will set the threshold value.

In an exemplary embodiment, categories of molecular variants in the SCN1A gene that are to be installed into transgenic zebrafish are first identified. In this first step, a set of 50 SCN1A variants (22 pathogenic, 22 benign and 6 VUS) are identified using a combination of parameters such as, e.g., literature review and expert opinion. Once the set of 50 variants is obtained, half of the established variants (11 pathogenic and 11 benign variants) are selected to provide a training set for examining the capacity of the F0 Crispinlet knock-in assay to detect variant pathogenicity in zebrafish embryos, while the other half (11 pathogenic and 11 benign variants) are used as a validation set and calculation of assay sensitivity and specificity (e.g. performance). The six remaining with VUS assignment status are screened using the system for their propensity to be above or below the predetermined threshold value. The number of variants selected is made due to the expected quantitative variability in the propensity to be pathogenic, wherein a minimum of 11 pathogenic and 11 benign variants enable reliable mapping of the dynamic range for a given functional assay (e.g., cell death assay). Although there will be a region where functionally abnormal assessment will be ambiguous, the region below this threshold will define the “window of pathogenicity” wherein a “functionally abnormal” assessment can be made. Each of the variants are individually tested for their effect on zebrafish embryo survivability.

In an exemplary embodiment, a guide gRNA is paired with a dhODN which instructs the cell's homologous repair machinery to make a genomic edit inserting a clinically-observed missense change. As a control for each locus, the same gRNAs are paired with an dhODN instructing for a synonymous coding change (a “viability” control). For example, the R406H variation in STXBP1 is a variant with well documented pathogenicity. When a particular number of embryos are injected for a STXBP1 F0 Crisplet study, only a certain number of those survive until day 15. This percent survival is likely to be due to high degree of biallelic conversion of most of the embryos to a somatic mosaicism status in which the R406H variation is present in both chromosome copies. For a second locus, the R388X protein truncation allele, embryo survival is observed to be nearly double that of R406H. The inability of the R388X injections to achieve similar lethality as the R406H variations may be due to the efficiencies of either the guide RNA or the donor homology to instruct the desired composition for repair. To minimize variability due to changes in composition of editing reagents, the above-mentioned synonymous-sequence dhODN is utilized, and provides for insertion of a variation that does not alter the amino acid sequence of the test protein (e.g., the clinical variant). In other words, the synonymous-sequence dhODN is expected to behave like a benign variation and the levels of embryo lethality resulting biallelic conversion therefrom in tissues is expected to be either absent or very low. The synonymous-sequence dhODN will therefore act as a signal normalization tool for each variant locus, providing a measure of all editing liabilities except the instructed conversion to a patient-observed genomic variation. If a high level of lethality is observed in the control animal, an alternative gRNA is selected and paired with new test and control dhODNs. Each variant is screened for effect on embryo lethality.

In an exemplary embodiment using the SCN1A clinical variant, each injection uses a morpholino targeting the duplication paralog for SCN1A, the scn1aa gene. To create a F0 crispinlet knock-in assay, gene editing is done in scn1ab while a morpholino is used to knock down scn1aa activity. The result is embryo lethality when an dhODN efficiently instructs for insertion of a pathogenic variant into scn1ab and the morpholino eliminates expression from the scn1aa paralog. In some embodiments, for a given F0 Crisplet knock-in assays, approximately 300 embryos are injected and scored at day 1 to day 10 for embryo survival rates to produce “training set data” for use in machine learning systems, including but not limited to those described herein. Once the training set data is acquired, the remaining half “hold-out” of the established variants (11 pathogenic and 11 benign) are screened similarly. The second step is considered achieved when the earliest day of lethality assessment is found to provide better than 90% sensitivity and 90% specificity collectively, and a threshold is determined for identifying a window of pathogenicity. In some embodiments, a ROC curve is used to determine how well a given lethality effect (e.g., 20% embryo survival) correlates with known pathology. In such embodiments, there will be an inflection point in the ROC that indicates the optimal lethality cut-off point that captures the most pathologies (e.g., it may be all sets of embryos with 25% or less survival are in the window of pathogenicity), without capturing benign clinical variants.

This training set data, and other data generated as described in this example and otherwise herein, can then be used with the machine learning algorithms as described herein to determine and/or predict pathogenicity (or lack thereof) of variants of unknown significance (VUS). In some embodiments, other phenotypes such as hyperpigmentation and seizure behavior for quantification of pathogenicity can also be determined and combined/used with the data generated as described herein. For instance, in some embodiments, animals can be stressed to elicit a phenotype/lethality earlier, as morphants have been shown to be sensitive to hyperthermia. Hyperthermia stress can also provide a transcriptomic readout since general hyperthermia/fever are known to induce changes like increased expression of immune modulators (IL1-beta, IL-6, neuropeptide Y), altered ion channel kinetics, axonal conduction velocity, and/or over-activation of TRPV4-channels.

### Example 4. F0 Crisprin-seq: Functional Assessment Assay of NOTCH3 Variants in CADASIL

This disclosure also provides methods for identifying pathogenicity using transcriptome profiling. To determine variant pathogenicity using these methods, the transcriptome is profiled via RNAseq of CRISPR-edited F0 knock-ins in an assay referred to as “F0 Crisprin-seq” (FIG. 7A). Zebrafish embryos are injected with CRISPR/Cas9 editing reagents and examined as hatchlings for changes in RNA expression levels as determined using RNAseq data. RNAseq uses next-generation sequencing (NGS) to reveal the presence and quantity of RNA in a biological sample at a given moment, analyzing the continuously changing cellular transcriptome (i.e., the set of all RNA molecules, here specifically mRNA molecules, in one cell or a population of cells). Thus, RNAseq provides a measure of the total expression response of an animal. Applied to variant biology analysis, RNAseq comparison between wild type and a clinical variant can be used to observe changes in expression behavior that are specific to the variant condition. This F0 Crisprin-seq assay system can uncover the quantitative changes in the transcriptome that are specific to the severity of a variant pathogenicity. This transgenic animal system has been used to show that high levels of biallelic conversion in the STXBP1 gene result in significant embryo lethality (FIG. 7B). The “transcriptome response” in surviving F0 Crisprin variants is similarly likely to correlate to the strength of a variant's pathogenicity. Whole transcriptomic data can be derived from CRISPR/Cas9 editing reagents injected into zebrafish embryos (the “F0 Crisprin” population). Machine learning techniques, such as those described herein, are then deployed on the RNAseq data (a training data set) to identify the key transcriptional responses that are specific to variant pathogenicity. Thus, in some embodiments, changes in the transcriptome (i.e., mRNA transcript expression) corresponding to variant pathogenicity can be identified using the training set of established pathogenic and benign variants.

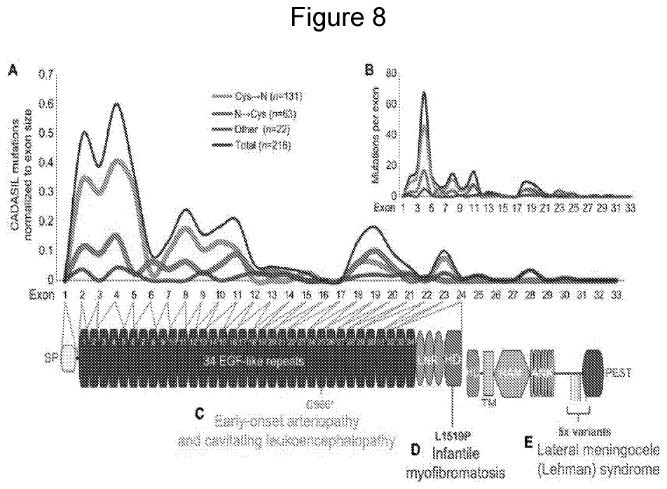

In this illustrative embodiment, variants in the NOTCH3 gene are studied using the F0 Crisprin-seq assay system. In 1996, NOTCH3 was discovered as the gene responsible for leukoencephalopathy/non-amyloidogenic angiopathy (PMID: 8878478). Since then many variations have been discovered in the epidermal-growth-factor-like (EGF-like) repeats of the extracellular domain (PMID: 9388399, 9329692, 15995828). According to a search in LitVar (PMID: 29762787) there have been 139 publications covering the top 10 pathogenic variants, all of which occur in the extracellular domain and are linked to cerebral autosomal dominant arteriopathy with subcortical infarcts and leukoencephalopathy (CADASIL). The frequency of pathogenic NOTCH3 variants in the general population occurs as high as 1 in 300 individuals (PMID: 30032161). A range of phenotypes have been observed with high severity being specific to the first 1-6 EGF repeats whereas variants in the 7 to 34 EGF repeats are known to be less severe (PMID: 30032161). The NOTCH3 receptor has two domains that contribute to unique pleiotropic disease effects (FIG. 8). In the cytoplasmic domain, a set of gain-of-function mutations lead to Leman's syndrome. Two additional variations have been observed and lead the conditions of early onset arteriopathy with cavitating leukodystrophy (C966X) and lateral meningocele syndrome (L1519P).

In the NOTCH3 gene, there are a wide variety of variations, many of which are associated with CADASIL (positive test) and non-CADASIL disease variations (negative test). CADASIL Was initially thought to be caused by autosomal-dominant, gain-of-function variants in NOTCH3—specifically, most variations involve a change in the balance of extracellular cysteine residues (Joutel A. Human Mutation. 2013 November; 34(11)). Yet recently CADASIL-associated variants were found that do not alter cysteine ratios. This draws into question a cystine-mediated gain-of-function mechanism (Muino et al. Int J Mol Sci. 2017 Sep. 13; 18(9)). As a result, loss-of-function alleles causing haploinsufficiency remain controversial in CADASIL. Some researchers have suspected non-cysteine altering variations may instead be associated with alternative disease outcomes such as diabetic retinopathy (Liu et al. J Vasc Res. 2018; 55(5):308-318). Applied to NOTCH3, prior work in NOTCH3 indicates that both patient-derived CADASIL vascular tissues and transgenic NOTCH3 R169C mice both exhibit the ER stress response underlying the vasculopathy of CADASIL (Neves, et al. JCI Insight, 4(23): e131344 (2019) (PMID: 31647781)). Vascular tissue from a NOTCH3 C475T (R133C) patient shows differential expression in 11 genes involved in protein degradation and folding, contraction of VSMCs, and cellular stress (Ihalainen, et al. Mol. Med. 13(5-6): 305 (2007) (PMID: 17622327)). In another study, the lower proliferative rate of vascular smooth muscle cells (VCMSs) of CADASIL derived tissue was correlated increased expression of transforming growth factor-β (TGFβ) gene expression and when a TGFβ-neutralizing antibody was added, the proliferation rate of CADASIL-derived VSMCs returned to normal (Panahi, et al. J. Cell. Mol. Med., 22(6): 3016-3024 (2018) (PMID: 29536621)). The vascular stress response in NOTCH3 has also been studied (Tang, et al. Mol. Biol. Rep., 44(3): 273-280 (2017) (PMID:28601945)). Thus, a spectrum of phenotypic consequences can result following expression of NOTCH3 variants. Modeling a diverse set of variants in the NOTCH3 receptor, including but not limited to those described herein, will be needed to better understand CADASIL and develop a companion diagnostic specific to the disease. By examining the transcriptome response between the positive and negative groups using the assays described herein, the pathway response specific to inducing CADASIL can be determined.

There are four notch receptors in humans, of which three (NOTCH1, NOTCH2 and NOTCH3) are recognized as involved in disease (OMIM). The NOTCH1 receptor is associated with Adams-Oliver syndrome and aortic valve disease. NOTCH2 is associated with Alagille syndrome and Hajdu-Cheney syndrome. On sequence similarity, both NOTCH1 and NOTCH2 have 61% sequence similarity to NOTCH3. In zebrafish, direct reciprocal orthologs exist for NOTCH3 (notch3) and NOTCH2 (notch2), while the NOTCH1 gene is represented as a duplicated gene (notch1a and notch1b). The sequence similarity between human NOTCH3 and zebrafish notch3 is 64%. In 2013, variations in zebrafish notch3 cytoplasmic domain were discovered to be associated with vascular defects (PMID: 23720232). As a result, the zebrafish notch3 gene has sufficient conservation of sequence and function that it is a good candidate for being an appropriate animal model of CADASIL.

To profile the transcriptome response related to NOTCH3 variant pathogenicity, the transcriptome is profiled via RNAseq of CRISPR-edited NOTCH3 variant F0 knock-ins using the F0 Crisprin-seq assay. Zebrafish embryos are injected with CRISPR/Cas9 editing reagents and examined as hatchlings for changes in RNA expression levels. The RNAseq profile for a wild-type strain is determined and compared to the RNAseq profile of a strain with a pathogenic stxbp1 R406C variant installed (FIG. 7B). To capture the expected diversity of gene expression changes that may be associated with such pathological responses, RNAseq (transcriptome profiling) is used to monitor expected changes in gene expression. While some NOTCH3 variants may be lethal to the transgenic animals generated as described herein, analysis of the transcriptome response in surviving F0 Crisprin variants will correspond to the pathogenicity of particular NOTCH3 variants. In some embodiments, RNA seq data (transcriptome profile, transcriptome feature) from at least one animal expressing a pathogenic variant and from at least one animal expressing a benign variant is needed in order to uncover which signaling pathways are being adjusted with gene expression response. Machine learning techniques are then deployed on the RNAseq data (transcriptome profile, transcriptome feature) to identify the key transcriptional responses that are specific to variant pathogenicity.

In one embodiment, a set of 25 established pathogenic and 25 established benign NOTCH3 variants is identified as a “training set” of data. CRISPR/Cas9 gene editing techniques are used for each identified NOTCH3 variant to create populations of F0 embryos with high levels of biallelic conversion. RNAseq of each embryo is carried out to create a dataset (e.g., inputs) of NOTCH3 variant-specific transcriptional responses (“transcriptome profiles”, “transcriptome features”). Technical difficulties can occur and can be tolerated for up to 49% of the targeted loci. PCR-confirmed transgenesis can then be used to yield sets of NOTCH3 variant specific RNAseq profiles (“transcriptome profiles”, “transcriptome features”) for at least 51% of the targeted NOTCH3 variants. In this manner, pathogenic transcriptome features (e.g., profiles) for the NOTCH3 variants can be determined.

In another embodiment, a set of 44 NOTCH3 variants, including 22 pathogenic and 22 benign variants are selected for use in determining the bounds for the window of pathogenicity (e.g. pre-determined threshold). Half of these variants are to be used as training set and the remaining half are used as validation set and calculation of assay sensitivity and specificity. Selection of variants will be performed, e.g., by combining published data (ClinVar and LitVar) with expert review. Once the set of 44 variants is identified, each variant is used for a set of F0 Crisprin injections into zebrafish embryos. On day 5 post injection, PCR is used to confirm if appreciable levels of genomic conversion have occurred in a subpopulation of embryos. At least 10% biallelic conversion will be necessary for passing the PCR test. For injection sets passing the PCR-based conversion test, RNA is harvested from remaining embryos and RNAseq data is obtained. The RNAseq data from the 44 variants with established benign and pathogenic behavior is then used as “training” data for the identification of transcriptome features (e.g., profiles) on F0 Crispin populations. Training involves splitting the variants into a “test” set (11 pathogenic variants and 11 benign variants) and “validation” set (11 pathogenic variants and 11 benign variants). The choice of 11 in each test set conform to the recent mandate proposed by experts involved in setting up the ACMG-AMP guidelines (https://www.biorxiv.org/content/10.1101/709428v2.full). Because of expected quantitative variability in the propensity to be pathogenic, a minimum of 11 pathogenic and 11 benign variants are recommended to enable reliable mapping of the dynamic range for a given functional assay.

Machine learning (ML) techniques are then deployed on the RNAseq data for clustering, classification, and differential expression analysis. Dimension reduction methods such as PCA or UMAP will be used to get a low dimensional representation of the RNA expression data. Linear or nonlinear classification algorithms (such as Linear discriminant analysis, support vector machine) will be trained on the dimension-reduced training set and tested on the validation set. Specificity and sensitivity are calculated on the validation set of 11 pathogenic and 11 benign variants. In addition, statistical analysis of differential expression, especially variant-specific transcriptome responses, are expected to reveal CADASIL-specific signaling pathways. RNAseq of variant-installed animal models can be used to identify CADASIL-associated gene expression activity. A common response (i.e., transcriptome features, transcriptome profiles) between all NOTCH3 pathogenic variants that are absent in benign variants are sought, i.e., this assay can be used to identify one or more sets of gene expression response pathways (i.e., transcriptome features, transcriptome profiles) specific to NOTCH3 variant pathogenicity.

### Example 5. Using Ensemble Machine Learning Algorithms to Aggregate Features from Multiple Sources and Functional Assays for Classifying Clinical Variants of Unknown Significance as to Pathogenicity

Provided herein is an ensemble classification model and method aggregating multiple data sources for identifying clinical variants of unknown significance as pathogenic, likely pathogenic, benign, or likely benign. The various phenotype and transcriptome assays described herein, or that may be otherwise available, can be used in these systems.

Clinically relevant point mutations in genes are modeled in animals. C. elegans models are created using CRISPR/Cas9 or by other genome editing techniques such as MosSCI or extrachromosomal arrays. Zebrafish models are created by CRISPR/Cas9 site-directed mutagenesis of native orthologous genes (e.g., as in Example 3). Zebrafish models can be inherited germline edits of the native chromosome or somatic CRISPR-mediated mutants (crispants). Alternatively, knock-down (morpholino) or knock-out lines can be rescued by human mRNA or expression plasmids to create animals expressing clinically relevant proteins.

Animal models are characterized using multiple phenotyping assays and/or transcriptome assays. Behavioral, survival, developmental, morphological, and molecular phenotyping assays are used. Behavioral assays include; locomotion, chemosensation, mechanosensation, osmotic avoidance, thermal response, feeding, reproduction, learning, swimming, egg-laying, defecation, galvanotaxis, and circadian rhythms. (Hart, Anne C., ed. Behavior (Jul. 3, 2006), WormBook, ed. The C. elegans Research Community, WormBook, doi/10.1895/wormbook.1.87.1, http://www.wormbook.org.) Survival assays include; abiotic stress resistance (oxidative, hypoxic, hyperoxic, heat, cold, heavy metal, ER, UV, osmotic, etc.), pathogen resistance (gram-positive pathogenic bacteria, gram-negative pathogenic bacteria, fungi, parasites, etc.) and healthspan/lifespan (Park H H, Jung Y, Lee S V. Survival assays using Caenorhabditis elegans. Mol Cells. 2017 Feb. 28; 40(2): 90-99). Developmental assays include; growth rate, organ development (pharynx, vulva, cilia, etc.), fat storage, and dauer formation. Morphological assays include; length, width, area, volume, position, and optical density. Molecular assays include; gene expression measured by QPCR of specific targets, gene expression measured by fluorescent reporter, gene expression measured by whole transcriptome sequencing (transcriptome assays), protein expression, protein localization, and whole-genome sequencing. In these model organism functional assays, a ‘phenocopy’ of the human pathology of interest (animals exhibiting similar phenotypes to human) is not required, but rather pathogenicity in model organisms is discovered in the polyvariate space defined by features measured in these functional assays.

In this example, the EPG and movement data for C. elegans models as described in Examples 1 and 2 are combined with Zebrafish crispant phenotyping data. Zebrafish embryos are injected with Cas9, sgRNAs and donor homology oligonucleotides to create the benign variant V84I, and the pathogenic variants R122X and R406H in stxbp1a. Zebrafish stxbp1a knockout animals are non-viable. Pathogenic variants show decreased survival to adulthood, while benign variants are similar in survival to un-injected controls.

Additionally, features of clinically relevant variants are aggregated from other sources. Information includes variant frequency in healthy populations, in silico predictions, and human phenotype characterizations from clinical data. For STXBP1 related disorders phenotypic characterization may include medical terms such as Absent speech, Cerebral atrophy, Cerebral hypomyelination, Developmental regression, Loss of developmental milestones, EEG with burst suppression, Epileptic encephalopathy, Epileptic spasms, Generalized hypotonia, Generalized myoclonic seizures, Generalized tonic seizures, Generalized tonic-clonic seizures, Grand mal seizures, Hypoplasia of the corpus callosum, Hypsarrhythmia, Impaired horizontal smooth pursuit, Infantile encephalopathy, Intellectual disability, severe, Early and severe mental retardation, Neonatal onset, Severe global developmental delay, Spastic paraplegia, Spastic tetraplegia, Status epilepticus, Tremor, and Variable expressivity.

Multiple types of phenotypic data and/or transcriptome data from C. elegans and Zebrafish, data on population frequency, in silico predictions and human phenotype characterizations from known variants are used to train an ensemble machine learning model to create classification of pathogenicity for unknown variants.

This functional analysis is used in drug testing and screening. Animals containing genetic variations that are indicated to be pathogenic are treated with pharmacological compounds. The phenotypes and/or transcriptomes are measured before and after treatment. Restoration of the phenotype and/or transcriptome to the wild-type, meaning the degree in the decrease of the distance for a pathogenic variant cluster to the decision boundary, correlates with the likelihood the drug will be effective in treating the patient. Drug screening is performed on disease models to indicate that the drug would be effective for that disease. Drug screening is performed on knockout or pathogenic variant animals to indicate that the drug would be effective for patients with a mutation in that gene. Drug screening provides additional phenotypic and/or transcriptomic data that can be incorporated into the machine learning classification model.

### Example 6. Linear Discriminator Analysis (LDA) Applied for Pathogenicity Assessment of Clinical Variants in STXBP1

This example describes a machine learning classification model and its application for assessing the presence of pathogenicity in clinical variants of the STXBP1 epilepsy-associated gene.

Due to the rapid adoption of whole genome sequencing by clinicians in their clinical practice, large volumes of sequence variant data are being generated. Often the clinical deposits their genome sequencing data in publicly accessible databases. The ClinVar database (https://www.ncbi.nlm.nih.gov/clinvar) is the most common repository for variant information in disease-associated genes. The current types of variant submission in ClinVar range from large genomic deletions and translocations to single nucleotide variants. There are a variety of classifications of clinical impact, but the 5 most frequent assessments are Benign (B), Likely Benign (LB), Variants of Uncertain Significance (VUS), Likely Pathogenic (LP) and Pathogenic (P). Most challenging to interpret for clinical impact are the single nucleotide variations occurring in the coding sequence which cause amino acid substitutions or missense variations. Often the sequence context alone is insufficient for conclusive assessment. Association of a genetic variation with disease prevalence in a related family cohort can provide indication of pathogenicity. Alternatively, the observation of a new (de novo) variation in multiple patients can also provide associative indication of pathogenicity. Yet often a variation discovered in a disease-associated gene is novel to a single patient and its causality for pathogenicity remains uncertain so the variant is labeled a VUS. The amount of missense VUS in ClinVar is rapidly growing at a rate that is much faster than the other assessment (P, LP, LB and B). As a result, the level of uncertainty for disease association is expanding at an alarming rate. In STXBP1, the ratio of missense variants as VUS (71×) to all of the missense variants observed (155×) is 46%. Functional tests can have a significant impact on assessment of pathogenicity in a variant. If a gene function test were applied to all of the VUS in STXBP1 and it lead to resolution of half of the VUS as pathogenic (35×), then added to the existing pathogenic variants (62× as P and LP), the diagnostic yield for pathogenicity assessment in this gene would increase by 56%.

Creation of a gene-humanized nematode expressing the human STXBP1 coding sequence is described herein. Functional testing in an intact animal model provides assessment of abnormal behavior for a clinical variant in a living animal context. The C. elegans animal model is chosen as the backdrop for the assessment of clinical variants in STXBP1. The first step in creating a test for assessing function of a variant function in C. elegans is to create a gene-swap humanized animal (FIG. 9a). The coding sequence for the most abundantly expressed isoform of STXBP1 (isoform a) was harvested from UniProt database (https://www.uniprot.org/). The sequence was codon-optimized for transgene expression in C. elegans. Three synthetic introns were introduced and the sequence was further optimized for enabling splicing specific only to the introduced introns. The sequence was introduced into the native unc-18 locus using CRISPR gene editing plasmid template. Two sgRNA sites were selected to be occurring within the first exon of unc-18. A plasmid was designed to provide donor homology sequences flank the outside edges of the two sgRNA cut sites. Within the plasmid sequence and between the two donor homology sequences, the codon-optimized hSTXBP1 sequence was introduced using standard molecular cloning techniques. Care was taken to design for elimination of the sgRNA sites to avoid recutting of the edited locus. The plasmid along with Cas9 and sgRNAs was injected into the hermaphrodite gonad to elicit transgene insertion. Animals were harvested by antibiotic selection for transgene insertion and homozygous animals were harvested. Verification of desired edit was confirmed by PCR followed by DNA sequencing. Once a confirmed transgenic animal was obtained an additional round of transgenesis was performed to remove the antibiotic selection. A repeat round of rtPCR and DNA sequencing was performed to confirm desired animal composition has been obtained (the “hSTXBP1-WT”).

Using the hSTXBP1-WT transgenic animal, a set of 57 clinical variations were installed using a modified CRISPR gene-editing procedure. Seventeen were selected as Benign from variants either labeled as benign in ClinVar or as variants seen in high frequency in healthy populations (https://gnomad.broadinstitute.org/). Twenty-two were selected as Pathogenic from variants that were labeled as pathogenic in ClinVar and/or observed multiple independent times in the literature (https://www.ncbi.nlm.nih.gov/CBBresearch/Lu/Demo/LitVar/). The remaining 18 variants were harvested from variants annotated as VUS in ClinVar. For each transgenesis an oligonucleotide was used as the donor homology template. For instance, to insert the R406H pathogenic variation, a set of sgRNA were selected to flank the locus of interest. A donor homology oligonucleotide (dhODN) was designed to have at least 35 base pairs of homology on the outside ends of the cut sites. In the interval between the cut sites, the DNA was recoded with a combination of amino acid changing and silent mutations such that a R406H change would be made and the new edited sequence would avoid recutting with the sgRNA sites. The dhODN was co-injected into the hermaphrodite gonad with the appropriate sgRNAs and Cas9 and rol-6 co-CRISPR reagents. Animals were harvested for transgene insertion and homozygous animals were harvested. Verification of desired edit was confirmed by rtPCR followed by DNA sequencing.

Measurement of feature sets for observing phenotypic anomalies in clinical variant animals. A wide spectrum of aberrant phenotypic behaviors were observed for each transgenic animal. Deep phenotyping dataset were harvested in electrophysiology using a screen chip apparatus, and in solid and liquid growth media formats using the MBF and worm-scanner apparatus. For the activity, behavior and morphometrics on solid media with the MBF instrument, a set of 26 features were harvested for each transgenic animal strain type.

Creation of the LDA plot and quantitation of pathogenic anomalies in STXBP1. both individual worm and population data were analyzed by Principal Component Analysis (PCA) to identify the key subsets of features with coordinated influence. A Linear Discriminator Analysis (LDA) was applied to develop a classification landscape for separating benign from pathogenic behavior using the known pathogenic and benign variants as training constraints on the LDA algorithm (FIG. 9b; Red (“Pathogenic”) and green and blue (“Benign and WT”))). Once the classifier algorithm was established, the VUS were analyzed similarly and overlaid on the LDA plot (FIG. 9b; grey (“VUS”)).

Use of a training data set for determining a diagnostic cut off for pathogenicity in STXBP1. The magnitude of the radial vectors (distance) emanating to each point from the hSTXBP1-WT control (FIG. 9b; blue (“WT”) to each of the other variants was harvested and tabulated. A Receiver Operator Curve (ROC) was generated for the magnitude values of the known benign and pathogenic variants. An optimal cutoff was determined at an integral value with the maximal Accuracy and F1 score. A sensitivity of 95% and specificity of 71% was obtained. The Positive Predictive Value (PPV) of 0.808 and Negative Predictive Value (PPV) of 0.923 were obtained.

Assessment of abnormal behavior in VUS of STXBP1. Using the optimal cutoff as determined from the ROC curve, a region of exclusion for pathogenicity was applied across the VUS (FIG. 9c; grey box). Ten of the VUS were assessed as Benign, while eight of the VUS were assessed to be Pathogenic. The overall assessment yield provided 44% of the VUS as pathogenic (FIG. 9d). If this yield extrapolates to the entire VUS in STXBP1, the diagnostic yield for STXBP1-associated epilepsy is likely to be increased by 30%.

We claim:

1. A computer-implemented method comprising:
a) obtaining, by one or more processors, a data set comprising measured phenotype features of a transgenic organism expressing a human clinical variant, wherein the phenotype features are from a population of clinical variants, wherein the clinical variants are labeled with a diagnostic indicator of pathogenic or benign for a specific human disease;
b) selecting a subset of the measured phenotype features for inputs into a machine learning system, wherein the subset includes at least four phenotype features and the diagnostic indicator for the clinical variants;
c) randomly partitioning the data set in training data and validation data; and,
d) generating a classifier model using a machine learning system based on the training data and the subset of inputs, wherein each input has an associated weight, and wherein the classifier model provides binary outcomes selected from pathogenic or likely pathogenic above a threshold value or benign or likely benign below a threshold value; optionally wherein one or both of the threshold values are pre-determined.

2. A method, in a computer implemented system comprising at least one processor and at least one memory, the at least one memory comprising instructions executed by the at least one processor to cause the at least one processor to implement one or more classifier models to predict pathogenicity for a clinical variant of a human disease, comprising:
a) obtaining measured phenotype features of a transgenic organism expressing the human clinical variant;
b) classifying the clinical variant into a pathogenicity category of pathogenic or likely pathogenic using a first classifier model, wherein the first classifier model is generated by a machine learning system using a first training data set that comprises phenotype features from the transgenic organism of a panel of at least four phenotype features from a population of clinical variants, wherein the clinical variants are labeled with a diagnostic indicator of pathogenic or benign for the human disease, wherein the first classifier model classifies the clinical variant in a pathogenicity category of pathogenic or likely pathogenic using input variables of the measured phenotype features of a panel of phenotype features from the transgenic organism when an output of the first classifier model is above a threshold value, optionally wherein the threshold value is predetermined; and,
c) optionally providing notification to a user for patient testing when the clinical variant is predicted to be pathogenic or likely pathogenic for a human disease.

3. A computer-implemented method for using a classifier model to predict pathogenicity for a clinical variant of a human disease comprising:
a) obtaining, by one or more processors, a data set comprising measured phenotype features of a transgenic organism expressing a human clinical variant, wherein the phenotype features are from a population of human clinical variants, wherein the human clinical variants are labeled with a diagnostic indicator of pathogenic or benign for a specific human disease;
b) selecting a subset of the measured phenotype features for inputs into a machine learning system, wherein the subset includes at least four phenotype features and the diagnostic indicator for the human clinical variants;
c) randomly partitioning the data set into training data and validation data;
d) generating a classifier model using a machine learning system based on the training data and the subset of inputs, wherein each input has an associated weight, and wherein the classifier model provides binary outcomes selected from pathogenic or likely pathogenic above a threshold or benign or likely benign below the threshold; and,
e) obtaining at least four measured phenotype features of a transgenic organism expressing at least one test human clinical variant;
f) classifies the test clinical variant in a pathogenicity category of pathogenic or likely pathogenic using input variables of the measured phenotype features of a panel of phenotype features from the transgenic organism and the classifier model of d) when an output of the classifier model is above a threshold value; and,
f) optionally providing notification to a patient expressing the clinical variant(s) when the clinical variant is predicted to be pathogenic or likely pathogenic for a human disease.

4. The method of any one of claims 1-3, wherein the diagnostic indicator is selected from pathogenic, likely pathogenic, likely benign and benign.

5. The method of any preceding claim, wherein the human disease is selected from epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemia, xeroderma pigmentosum, Fanconi's anemia, retinitis pigmentosa, ataxia telangiectasia, Bloom's syndrome, retinoblastoma, or Tay-Sachs disease.

6. The method of any preceding claim, wherein the human disease is selected from the group consisting of neuromuscular, epilepsy, ataxia, dystonia, neurodegeneration, cancer, and a metabolic disease or condition.

7. The method of any preceding claim, wherein the at least one test clinical variant is a variant of unknown or uncertain significance or unassigned.

8. The method of any preceding claim, wherein the transgenic organism is a nematode or zebrafish.

9. The method of any preceding claim, wherein the phenotype features are measured in an electropharyngeogram (EPG) assay, morphology and/or movement phenotype assay, or a gene expression profile, lethality, incidence of males, axonal outgrowth, or synaptic transmission assay.

10. The method of any preceding claim, wherein the phenotype features are selected from pharyngeal pumping duration, inter-pump interval, pumping frequency, peak amplitude of different pump components, speed, forward vs. reverse travel, curling, length, width, lethality, attenuation, bending angle-mid-point asymmetry, maximum amplitude (um), self-contact distance, mean amplitude (um), body wave number, area, dynamic amplitude (stretch), center point speed (um/s), center point trajectory/time, peristaltic speed (um/s), absolute peristaltic track length/time, activity, brush stroke, length, reverse swim, curling, fit, swimming speed, wave initiation rate, wavelength, width, proportion time forward, proportion time reverse, straight-line speed, forward speed, and reverse speed.

11. The method of any preceding claim, wherein the first training data comprises values from a panel of at least five phenotype features.

12. The method of any preceding claim, wherein the first training data further comprises patient phenotype, patient drug response, or phenotype in a second transgenic organism expressing the human clinical variant, wherein the second transgenic organism is selected from frog oocyte, nematode or zebrafish, fly or rodent or iPSC cells.

13. The method of claim 12 wherein the transgenic organism and the second transgenic organism are different, optionally wherein the transgenic organism is a nematode and the second transgenic organism is zebrafish.

14. The method of any preceding claim, wherein the input variables comprise measured phenotype features from a panel of at least five phenotype features, optionally about six to about eight phenotype features, about nine to about 15 phenotype features, or about 16 to about 30 phenotype features.

15. The method of any preceding claim, wherein the machine learning system further comprises iteratively regenerating the first classifier model by training the first classifier model with new training data to improve the performance of the first classifier model.

16. The method of any preceding claim, wherein the first classifier model comprises a support vector machine, a decision tree, a random forest, a neural network, a deep learning neural network, pattern recognition, or a logistic regression algorithm.

17. The method of claim 1, further comprising:
(1) obtaining one or more test results from the diagnostic testing which confirm or deny the presence of the human disease;
(2) incorporating the one or more test results into the first training data for further training of the first classifier model of the machine learning system; and,
(3) generating an improved first classifier model by the machine learning system.

18. The method of any preceding claim, wherein the threshold is determined used performance of the classifier model as measured by sensitivity and specificity, optionally wherein the threshold value is determined based on a specificity of at least about 0.70.

19. The method of any preceding claim wherein the transgenic organism expresses the human clinical variant following modification to create the human clinical variant in the genome of the transgenic organism, optionally using CRISPR, and/or replacing the naturally-occurring coding sequence of the transgenic organism with a modified coding sequence; optionally wherein the presence of the clinical variant in the transgenic organism(s) is confirmed by nucleotide sequencing.

20. A computer-implemented method comprising:
a) obtaining, by one or more processors, a data set comprising measured transcriptome features of a transgenic organism expressing at least one human clinical variant, wherein the transcriptome features are from a population of clinical variants, wherein the clinical variants are labeled with a diagnostic indicator of pathogenic or benign for a specific human disease;
b) selecting a subset of the measured transcriptome features for inputs into a machine learning system, wherein the subset includes transcriptome features and the diagnostic indicator for the clinical variants;
c) randomly partitioning the data set in training data and validation data; and,
d) generating a classifier model using a machine learning system based on the training data and the subset of inputs, wherein each input has an associated weight, and wherein the classifier model provides binary outcomes selected from pathogenic or likely pathogenic above a pre-determined threshold or benign or likely benign below a pre-determined threshold.

21. A method, in a computer implemented system comprising at least one processor and at least one memory, the at least one memory comprising instructions executed by the at least one processor to cause the at least one processor to implement one or more classifier models to predict pathogenicity for a clinical variant of a human disease, comprising:
a) obtaining measured transcriptome features of a transgenic organism expressing the human clinical variant;
b) classifying the clinical variant into a pathogenicity category of pathogenic or likely pathogenic using a first classifier model, wherein the first classifier model is generated by a machine learning system using a first training data set that comprises transcriptome features from the transgenic organism of a panel of transcriptome features from a population of clinical variants, wherein the clinical variants are labeled with a diagnostic indicator of pathogenic or benign for the human disease, wherein the first classifier model classifies the clinical variant in a pathogenicity category of pathogenic or likely pathogenic using input variables of the measured transcriptome features of a panel of transcriptome features from the transgenic organism when an output of the first classifier model is above a predetermined threshold; and,
c) providing notification to a user for patient testing when the clinical variant is predicted to be pathogenic or likely pathogenic for a human disease.

22. The method of claim 20 or 21, wherein the diagnostic indicator is selected from the group consisting of pathogenic, likely pathogenic, likely benign and benign.

23. The method of any one of claims 20-22, wherein the human disease is selected from the group consisting of epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, or Tay-Sachs disease.

24. The method of any one of claims 20-22, wherein the human disease is selected from the group consisting of neuromuscular, epilepsy, ataxia, dystonia, neurodegeneration, cancer, and a metabolic disease or condition.

25. The method of any one of claims 20-24, wherein the clinical variant is a variant of unknown or uncertain significance or unassigned.

26. The method of any one of claims 20-25, wherein the transgenic organism is a nematode or zebrafish.

27. The method of any one of claims 20-26, wherein the first training data comprises values from a panel of transcriptome features.

28. The method of any one of claims 20-27, wherein the first training data further comprises patient transcriptome features, patient drug response, or transcriptome features in a second transgenic organism expressing the human clinical variant, wherein the second transgenic organism is selected from frog oocyte, fly or rodent.

29. The method of any one of claims 20-28, wherein the input variables comprise measured transcriptome features from a panel transcriptome features.

30. The method of any one of claims 20-29, wherein the machine learning system further comprises iteratively regenerating the first classifier model by training the first classifier model with new training data to improve the performance of the first classifier model.

31. The method of any one of claims 20-30, wherein the first classifier model comprises a support vector machine, a decision tree, a random forest, a neural network, a deep learning neural network, pattern recognition, or a logistic regression algorithm.

32. The method of any one of claims 20-31, further comprising: (1) obtaining one or more test results from the diagnostic testing which confirm or deny the presence of the human disease; (2) incorporating the one or more test results into the first training data for further training of the first classifier model of the machine learning system; and, (3) generating an improved first classifier model by the machine learning system.

33. The method of claim 1-3 or 20 wherein the classifier model is generated using a machine learning system based on the training data and the subset of inputs, each of which include measured phenotype features and measured transcriptome features.

34. The method of claim 2, 3 or 20 wherein the first classifier model classifies the clinical variant in a pathogenicity category of pathogenic or likely pathogenic using input variables of measured phenotype features and measured transcriptome features.

35. A method of any preceding claim wherein the classifier uses one threshold value for discriminating pathogenic or likely pathogenic from benign or likely benign.

36. A method of claim 30 wherein a range of threshold values is selected from the group consisting of direct outputs from either a radial or linear classifier, cartesian coordinates from an origin on dimension reduced plots, and composite euclidean vector magnitudes from multidimensional feature sets.

37. A method of claim 35 or 36 wherein the threshold value is determined by Receiver Operator Curve (ROC) curve analysis.

38. A method of any preceding claim wherein the classifier creates two threshold values using Firth logistic regression.

39. A method of claim 38 wherein the threshold values comprise a lower threshold value and a upper threshold value, wherein the lower threshold is the maximum threshold value for a clinical variant being identified as benign and the upper threshold value is the minimal threshold for being identified as pathogenic.
